(19) United States
(12) Patent Application Publication
(10) Pub. No.: US 2024/0248396 A1
(43) Pub. Date: Jul. 25, 2024

(54) XANTHENE COMPOUND, RESIN COMPOSITION, CURED OBJECT, METHOD FOR PRODUCING CURED OBJECT, ORGANIC EL DISPLAY DEVICE, AND DISPLAY DEVICE

(71) Applicant: TORAY INDUSTRIES, INC., Tokyo (JP)

(72) Inventors: Yusuke KOMORI, Otsu-shi (JP); Hiroki NISHIOKA, Otsu-shi (JP); Kazuto MIYOSHI, Otsu-shi (JP)

(73) Assignee: TORAY INDUSTRIES, INC., Tokyo (JP)

(21) Appl. No.: 18/571,832

(22) PCT Filed: Jul. 29, 2022
(86) PCT No.: PCT/JP2022/029310

§ 371 Date: Dec. 19, 2023

(30) Foreign Application Priority Data
Aug. 6, 2021 (JP) 2021-129709
Feb. 21, 2022 (JP) 2022-024575

(51) Int. Cl. G03F 7/004; C07D 311/82; C08K 5/18; G03F 7/022; H10K 59/122; H10K 59/80

(52) U.S. Cl. CPC G03F 7/0045; C07D 311/82; C08K 5/18; G03F 7/0226; H10K 59/122; H10K 59/8792

(57) ABSTRACT

An object of the present invention is to provide a xanthene compound having high heat resistance and capable of shielding light up to a long-wavelength region of visible light as compared to a conventional xanthene compound. In order to solve the above problem, a xanthene compound (b) of the present invention is a xanthene compound (b) represented by Formula (1).

### TECHNICAL FIELD

The present invention relates to a xanthene compound, a resin composition using the xanthene compound, an organic EL display device using the resin composition, and the like.

### BACKGROUND ART

Organic electroluminescence (hereinafter referred to as organic EL) display device has been used in display device having a thin display, such as smartphones, tablet personal computers, and televisions, to develop many products. In general, an organic EL display device has a drive circuit, a planarization layer, a first electrode, an insulation layer, a light-emitting layer, and a second electrode that are placed over a substrate. The organic EL display device can emit light when a voltage is applied between the first electrode and the second electrode facing to each other. As a material for a planarization layer or an insulation layer among the above-described components, photosensitive resin compositions are generally used that can be patterned by ultraviolet irradiation. Among the photosensitive resin compositions, photosensitive resin compositions in which a polyimide-based resin is used have high heat resistance of the resin and a small amount of gas components generated from the cured object, and therefore are suitably used from the viewpoint of obtaining a highly reliable organic EL display device.

In recent years, for the purpose of improving light extraction efficiency of an organic EL display device, thinning of a polarizing plate and a display device without a polarizing plate have been developed, and in order to improve contrast, it is required to lower the transmittance of visible light of an insulation layer and a planarization layer.

Examples of the technique of decreasing the transmittance of visible light in a cured object and increasing the blackness include a method of adding a colorant such as carbon black, an organic/inorganic pigment, or a dye to a resin composition as can be seen in black matrix materials for liquid crystal display devices and RGB paste materials.

Examples of the technique of increasing the blackness of a cured object in a positive photosensitive resin composition include a method of adding a quinone diazide compound and a black pigment to an alkali-soluble resin composed of a novolac resin and/or a vinyl polymer (see Patent Document 1), a method of adding a photosensitizer and a black pigment to a soluble polyimide (see Patent Document 2), and a method of adding a photosensitizer and yellow, red, and blue dyes and/or pigments to an alkali-soluble resin composed of a polyimide and/or a polyimide precursor (see Patent Document 3). As a dye having high heat resistance and a large molar absorption coefficient, for example, a xanthene compound is known (see Patent Documents 4 and 5).

### PRIOR ART DOCUMENTS

### Patent Documents

Patent Document 1: Japanese Patent Laid-open Publication No. H6-230215
Patent Document 2: Japanese Patent Laid-open Publication No. 2003-119381
Patent Document 3: Japanese Patent Laid-open Publication No. 2018-63433
Patent Document 4: Japanese Patent Laid-open Publication No. 2014-9330
Patent Document 5: Japanese Patent Laid-open Publication No. 2020-111627

### SUMMARY OF THE INVENTION

### Problems to be Solved by the Invention

A conventional xanthene compound has high heat resistance, but has a maximum absorption wavelength around 550 nm, so that the light shielding property particularly in a long-wavelength region of visible light is not sufficient.

### Solutions to the Problems

The present invention adopts the following constitution to solve the problem described above.

[1] A xanthene compound (b) represented by Formula (1):

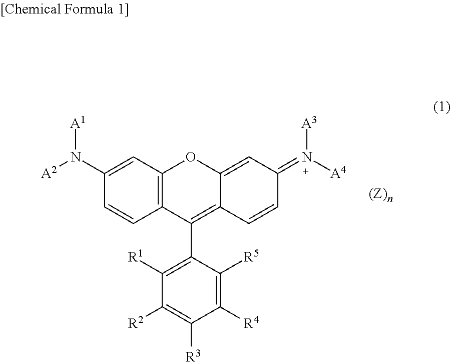

wherein A¹ to A⁴ each independently represent a hydrogen atom, an alkyl group having 1 to 10 carbon atoms, or an aryl group having 6 to 10 carbon atoms which may have an electron donating substituent, provided that, at least three of A¹ to A⁴ are the aryl groups having 6 to 10 carbon atoms which may have an electron donating substituent, and at least one of the aryl groups having 6 to 10 carbon atoms which may have an electron donating substituent has an electron donating substituent; R¹ to R⁴ each independently represent a hydrogen atom, a halogen atom, a hydroxyl group, an alkoxy group, —SO₃H, —SO₃⁻, —SO₃NR⁶R⁷, —COOH, —COO⁻, —COOR⁸, —CONR⁹R¹⁰, or a monovalent hydrocarbon group having 1 to 20 carbon atoms; R⁵ represents a hydrogen atom, —SO₃H, —SO₃⁻, —SO₃NR⁶R⁷, —COOH, —COO⁻, —COOR⁸, or —CONR⁹R¹⁰; R⁶ to R¹⁰each independently represent a monovalent hydrocarbon group having 1 to 20 carbon atoms; and Z represents an anionic compound, and n represents 0 or 1, provided that, the xanthene compound (b) represented by Formula (1) is charge neutral as a whole.
   [2] The xanthene compound (b) according to [1], in which a value of a substituent constant σₚ of Hammett equation of the electron donating substituent is −0.20 or less.
   [3] The xanthene compound (b) according to [1] or [2], in which n is 0 in the above Formula (1).
   [4] The xanthene compound (b) according to [1] or [2], in which n is 1 and Z is an aliphatic or aromatic sulfonate ion in the above Formula (1).
   [5] A resin composition containing the xanthene compound (b) according to any one of [1] to [4] and an alkali-soluble resin (a).
   [6] The resin composition according to [5], further containing a photosensitive compound (c).
   [7] The resin composition according to [6], in which the photosensitive compound (c) contains a quinone diazide compound.
   [8] The resin composition according to any one of [5] to [7], further containing a colorant (d-2) having a maximum absorption wavelength in any of a range of 490 nm or more and less than 580 nm at 350 to 800 nm.
   [9] The resin composition according to any one of [5] to [7], containing a xanthene compound (b1) in which n is 1 and Z is an organic anion in the above Formula (1) and an ionic dye (d10) forming an ion pair of organic ions, the organic anions being one kind.
   [10] The resin composition according to any one of [5] to [9], in which the alkali-soluble resin (a) contains one or more selected from the group consisting of a polyimide, a polyimide precursor, a polybenzoxazole, a polybenzoxazole precursor, a polyamide-imide, a polyamide-imide precursor, and a copolymer thereof.
   [11] The resin composition according to any one of [5] to [10], in which a total mass of all chlorine atoms and all bromine atoms contained in the resin composition is 150 ppm or less based on a total mass of solid contents of the resin composition.
   [12] A cured object obtained by curing the resin composition according to any one of [5] to [10].
   [13] A cured object containing a xanthene compound (b′) represented by Formula (2):

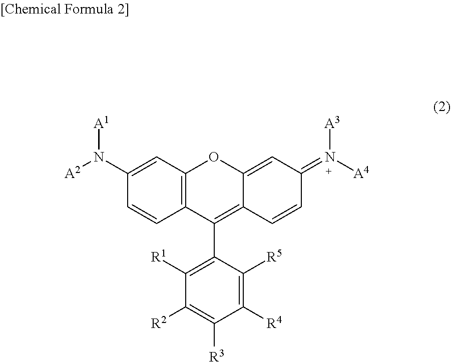

wherein A¹ to A⁴ each independently represent a hydrogen atom, an alkyl group having 1 to 10 carbon atoms, or an aryl group having 6 to 10 carbon atoms which may have an electron donating substituent, provided that, at least three of A¹ to A⁴ are the aryl groups having 6 to 10 carbon atoms which may have an electron donating substituent, and at least one of the aryl groups having 6 to 10 carbon atoms which may have an electron donating substituent has an electron donating substituent; R¹ to R⁴ each independently represent a hydrogen atom, a halogen atom, a hydroxyl group, an alkoxy group, —SO₃H, —SO₃⁻, —SO₃NR⁶R⁷, —COOH, —COO⁻, —COOR⁸, —CONR⁹R¹⁰, or a monovalent hydrocarbon group having 1 to 20 carbon atoms; R⁵ represents a hydrogen atom, —SO₃H, —SO₃⁻, —SO₃NR⁶R⁷, —COOH, —COO⁻, —COOR⁸, or —CONR⁹R¹⁰; and R⁶ to R¹⁰ each independently represent a monovalent hydrocarbon group having 1 to 20 carbon atoms, provided that, the xanthene compound (b′) represented by Formula (2) is charge neutral or cationic.
   [14] A method for producing a cured object, including the steps of: forming a resin film formed of the resin composition according to any one of [6] to [11] on a substrate; exposing the resin film; developing the exposed resin film; and subjecting the developed resin film to heat treatment.
   [15] The method for producing a cured object according to [14], in which in the step of exposing the resin film, a photomask used for exposure is a half-tone photomask having a light-transmitting portion, a light-shielding portion, and a semi-translucent portion, and a transmittance of the semi-translucent portion is 5% to 30% when a transmittance of the light-transmitting portion is taken as 100%.
   [16] An organic electroluminescence (EL) display device including: a substrate; a drive circuit; a planarization layer; a first electrode; an insulation layer; a light-emitting layer; and a second electrode, in which the drive circuit, the planarization layer, the first electrode, the insulation layer, the light-emitting layer, and the second electrode are placed over the substrate, and the planarization layer and/or the insulation layer includes the cured object according to [12] or [13].
   [17] The organic EL display device according to [16], in which the insulation layer includes the cured object, and an optical density of the insulation layer in visible light per film thickness of 1 μm is 0.5 to 1.5.
   [18] The organic EL display device according to [16] or [17], further including a color filter having a black matrix.
   [19] A display device including at least a metal wiring, the cured object according to [12] or [13], and a plurality of luminescent elements, wherein each of the luminescent elements includes a pair of electrode terminals on either one surface, the pair of electrode terminals are connected to a plurality of the metal wirings extending in the cured object, and the plurality of the metal wirings are configured to retain electrical insulation properties by the cured object.

### Effects of the Invention

There is provided a xanthene compound having high heat resistance and capable of shielding light up to a long-wavelength region of visible light as compared to a conventional xanthene compound.

### BRIEF DESCRIPTION OF THE DRAWINGS

FIG. 1 is a cross-sectional view of an example of an organic EL display device.

FIG. 2 is a cross-sectional view of an example of a display device.

### EMBODIMENTS OF THE INVENTION

An embodiment of the present invention will be described in detail.

<Xanthene Compound (b)>

A xanthene compound (b) of the present invention is a compound represented by Formula (1).

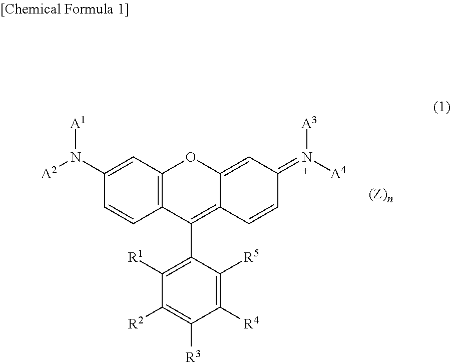

In Formula (1), A¹ to A⁴ each independently represent a hydrogen atom, an alkyl group having 1 to 10 carbon atoms, or an aryl group having 6 to 10 carbon atoms which may have an electron donating substituent. Provided that, at least three of A¹ to A⁴ are the aryl groups having 6 to 10 carbon atoms which may have an electron donating substituent, and at least one of the aryl groups having 6 to 10 carbon atoms which may have an electron donating substituent has an electron donating substituent. R¹ to R⁴ each independently represent a hydrogen atom, a halogen atom, a hydroxyl group, an alkoxy group, —SO₃H, —SO₃⁻, —SO₃NR⁶R⁷, —COOH, —COO⁻, —COOR⁸, —CONR⁹R¹⁰, or a monovalent hydrocarbon group having 1 to 20 carbon atoms. R⁵ represents a hydrogen atom, —SO₃H, —SO₃⁻, —SO₃NR⁶R⁷, —COOH, —COO⁻, —COOR⁸, or —CONR⁹R¹⁰. R⁶ to R¹⁰ each independently represent a monovalent hydrocarbon group having 1 to 20 carbon atoms. Z represents an anionic compound, and n represents 0 or 1. Provided that, the xanthene compound (b) represented by Formula (1) is charge neutral as a whole.

In the xanthene compound (b) of the present invention, since at least three of A¹ to A⁴ are the aryl groups having 6 to 10 carbon atoms which may have an electron donating substituent, and at least one of the aryl groups having 6 to 10 carbon atoms which may have an electron donating substituent has an electron donating substituent in Formula (1), the maximum absorption wavelength at 350 to 800 nm can be further lengthened than that of a xanthene compound not having such conditions.

Examples of the aryl group having 6 to 10 carbon atoms the aryl group having 6 to 10 carbon atoms which may have an electron donating substituent can include a phenyl group and a naphthyl group. In Formula (1), from the viewpoint that the maximum absorption wavelength at 350 to 800 nm can be further lengthened, all four of A¹ to A⁴ are preferably aryl groups.

At least one of the at least three aryl groups having 6 to 10 carbon atoms which may have an electron donating substituent has an electron donating substituent. When the aryl group on the nitrogen atom in Formula (1) has an electron donating substituent, the maximum absorption wavelength at 350 to 800 nm of the xanthene compound (b) can be further lengthened. The electron donating substituent is an atomic group that donates an electron to a substituted atomic group by the inductive effect and/or the resonance effect in the organic electron theory. Examples of the electron donating substituent include ones having a negative value as a value of a substituent constant σₚ of Hammett equation. The value of the substituent constant σₚ of Hammett equation can be cited from Kagaku Binran Kiso-Hen Revised 5th Edition (II, p. 380). Specific examples of the electron donating substituent can include an alkyl group (σₚ value of methyl group: −0.17), an alkoxy group ((p value of methoxy group: −0.27), an aryloxy group (σₚ value of —OC₆H₅: −0.32), a hydroxyl group (σₚ value of —OH: −0.37), an amino group (σₚ value of —NH₂: −0.66), and an alkylamino group (σₚ value of —N(CH₃)₂: −0.83).

From the viewpoint that the maximum absorption wavelength at 350 to 800 nm of the xanthene compound (b) can be lengthened, a value of a substituent constant σₚ of Hammett equation of the electron donating substituent is preferably −0.20 or less, preferably −0.25 or less, and still more preferably −0.30 or less. The lower limit of the value of the substituent constant σₚ of Hammett equation is not particularly limited, but is preferably −0.90 or more.

When three of A¹ to A⁴ are aryl groups having 6 to 10 carbon atoms which may have an electron donating substituent, it is preferable that two or more of the aryl groups having 6 to 10 carbon atoms which may have an electron donating substituent have the electron donating substituent, and it is more preferable that three aryl groups having 6 to 10 carbon atoms which may have an electron donating substituent have an electron donating substituent.

When four of A¹ to A⁴ are aryl groups having 6 to 10 carbon atoms which may have an electron donating substituent, it is preferable that two or more aryl groups having 6 to 10 carbon atoms which may have an electron donating substituent have an electron donating substituent, it is more preferable that three or more aryl groups having 6 to 10 carbon atoms which may have an electron donating substituent have an electron donating substituent, and it is still more preferable that four aryl groups having 6 to 10 carbon atoms which may have an electron donating substituent have an electron donating substituent.

The preferred substitution position of the electron donating substituent is preferably a para-position or an ortho-position with respect to a carbon atom bonded to the xanthene compound (b) via a nitrogen atom, and more preferably a para-position.

The aryl group having 6 to 10 carbon atoms which may have an electron donating substituent may have a substituent other than the above-described electron donating substituent. Examples of the substituent other than the electron donating substituent can include an aryl group, a halogen atom, and a monovalent group represented by —COORa, —OCORa, —SO₃⁻, or —SO₂Ra. However, since the compound represented by Formula (1) is charge neutral as a whole, when the aryl group having 6 to 10 carbon atoms has —SO₃⁻, the number of substitutions of —SO₃⁻ is 1, and R¹ to R⁵ have a neutral group. Ra represents an alkyl group. From the viewpoint of reducing the molecular weight of the xanthene compound (b) and increasing the ratio of the coloring component per unit mass, the substituent other than the electron donating substituent is preferably 20 or less carbon atoms and preferably 10 or less carbon atoms. From the same viewpoint, Ra preferably has 20 or less carbon atoms and preferably 10 or less carbon atoms. The sum of values of the substituent constant σₚ of Hammett equation for bonding to the aryl group having 6 to 10 carbon atoms which may have an electron donating substituent is preferably −0.20 or less.

A¹ and A² and/or A³ and A⁴, respectively, may be bonded to form a ring. As for these rings, a ring may be formed by a single bond or a bond via any atom of a nitrogen atom, an oxygen atom, or a sulfur atom. The ring to be formed in this case is preferably a 5-membered ring or a 6-membered ring. Examples of the ring to be formed can include a carbazole ring in which two aryl groups having 6 to 10 carbon atoms which may have an electron donating substituent are bonded via a single bond, and an indole ring in which an aryl group having 6 to 10 carbon atoms which may have an electron donating substituent and an alkyl group having 1 to 10 carbon atoms are bonded via a single bond.

R¹ to R⁴ each independently represent a hydrogen atom, a halogen atom, a hydroxyl group, an alkoxy group, —SO₃H, —SO₃⁻, —SO₃NR⁶R⁷, —COOH, —COO⁻, —COOR⁸, —CONR⁹R¹⁰, or a monovalent hydrocarbon group having 1 to 20 carbon atoms. R⁶ to R¹⁰ each independently represent a hydrocarbon group having 1 to 20 carbon atoms. Examples of the hydrocarbon group having 1 to 20 carbon atoms can include an alkyl group, a cycloalkyl group, and an aryl group.

R⁵ represents a hydrogen atom, —SO₃H, —SO₃⁻, —SO₃NR⁶R⁷, —COOH, —COO⁻, —COOR⁸, or —CONR⁹R¹⁰. R⁶ to R¹⁰ each independently represent a monovalent hydrocarbon group having 1 to 20 carbon atoms. From the viewpoint of enhancing the heat resistance, R⁵ is preferably a hydrogen atom, —SO₃H, —SO₃⁻, —SO₃NR⁶R⁷, —COOR⁸, or —CONR⁹R¹⁰, and more preferably —SO₃H, —SO₃⁻, —SO₃NR⁶R⁷, or —CONR⁹R¹⁰. When R⁵ is —SO₃NR⁶R⁷, from the viewpoint of enhancing the heat resistance, any one of R⁶ and R⁷ is preferably an aryl group, and R⁶ and R⁷ are more preferably an aryl group. When R⁵ is —CONR⁹R¹⁰, from the viewpoint of enhancing the heat resistance, any one of R⁹ and R¹⁰ is preferably an aryl group, and R⁹ and R¹⁰ are more preferably an aryl group.

Z represents an anionic compound. When the compound represented by Formula (1) has an anionic compound represented by Z, n is 1. The anionic compound may be either an inorganic anion or an organic anion, and can include a halide ion such as chlorine or bromine as an inorganic ion and a sulfonimide anion [(RSO₂)₂N]⁻, a borate anion (BR₄)⁻, and the like as an organic ion, in addition to the aliphatic or aromatic sulfonate ion and the aliphatic or aromatic carboxylate ion. R in the ionic formula is each independently a monovalent hydrocarbon group having 1 to 20 carbon atoms which may have a substituent and may have a heteroatom in the carbon chain. Examples of the substituent of R include an alkyl group having 1 to 10 carbon atoms, an aryl group having 1 to 10 carbon atoms, a halogen atom, a hydroxyl group, an alkoxy group, and an aryloxy group. Examples of the heteroatom include a nitrogen atom, an oxygen atom, and a halogen atom. From the viewpoint of suppressing deterioration of an electrode or a light-emitting layer in an organic EL display device when the cured object formed of the resin composition having the xanthene compound (b) is applied to the organic EL display device, in Formula (1), n is 1, and the anionic compound of Z is preferably an organic anion, preferably an aliphatic or aromatic sulfonate ion, an aliphatic or aromatic carboxylate ion, a sulfonimide anion, or a borate anion. From the viewpoint of improving the sensitivity and the viewpoint of reducing the residues when a resin composition containing an alkali-soluble resin (a) and a photosensitive compound (c) described below is obtained, in Formula (1), n is 1, Z is preferably an aliphatic or aromatic sulfonate ion or an aliphatic or aromatic carboxylate ion, and Z is more preferably an aliphatic or aromatic sulfonate ion. The aliphatic group is preferably a monovalent alkyl group having 1 to 20 carbon atoms, and examples thereof include a methyl group, an ethyl group, a propyl group, a butyl group, and a group in which a part of hydrogen atoms of these alkyl groups is substituted with a halogen atom. The aromatic group is preferably a monovalent aryl group having 1 to 20 carbon atoms, and examples thereof include a phenyl group, a tolyl group, an ethylphenyl group, a propylphenyl group, a butylphenyl group, and a dodecylphenyl group. From the viewpoint of improving the sensitivity by increasing the ratio of the coloring component per molecule and decreasing the addition amount of the ionic dye, the molecular weight of Z is preferably 1000 or less, preferably 700 or less, and still more preferably 300 or less. The lower limit of the molecular weight of Z is not particularly limited, and is preferably 1 or more and more preferably 100 or more.

n represents 0 or 1. Provided that, the compound represented by Formula (1) is charge neutral as a whole. The phrase “charge neutral” refers to a state that the positive charge number and the negative charge number of the compound represented by Formula (1) coincide with each other. Since the compound represented by Formula (1) is charge neutral as a whole, when R¹ to R⁵ contain an anion, only one of R¹ to R⁵ is —SO₃⁻ or —COO—. When only one of R¹ to R⁵ in the xanthene compound (b) is —SO₃⁻ or —COO— or when the aryl group having 6 to 10 carbon atoms has —SO₃⁻, since a counter anion exists in a substituent in the molecule, the compound represented by Formula (1) is charge neutral as a whole even if the compound does not have Z, and n is 0. On the other hand, when none of R¹ to R⁵ in the xanthene compound (b) contains an anion or when the aryl group having 6 to 10 carbon atoms does not have —SO₃⁻, since the compound represented by Formula (1) is charge neutral as a whole, n is 1. When n is 1, the compound represented by Formula (1) has Z. From the viewpoint of preventing halide ions from being mixed into the cured object formed of the resin composition having the xanthene compound (b), n is preferably 0 in Formula (1). Meanwhile, from the viewpoint of improving the sensitivity when a resin composition containing an alkali-soluble resin (a) and a photosensitive compound (c) described below is obtained, n is preferably 1.

The xanthene compound (b) preferably has a maximum absorption wavelength in any of a range of 580 nm or more and 700 nm or less at 350 to 800 nm. In general, although a xanthene compound in which the nitrogen atom is substituted with an alkyl group gives a red spectrum having a maximum absorption wavelength at 350 to 800 nm of about 550 nm, in the xanthene compound (b) represented by Formula (1), at least three of A¹ to A⁴ are the aryl groups having 6 to 10 carbon atoms which may have an electron donating substituent, and at least one of the aryl groups having 6 to 10 carbon atoms which may have an electron donating substituent has an electron donating substituent in Formula (1), so that the maximum absorption wavelength is lengthened, and a blue spectrum is obtained. The maximum absorption wavelength of the xanthene compound (b) is more preferably in any of a range of 590 nm or more and 700 nm or less and still more preferably in any of a range of 600 nm or more and 700 nm or less.

From the viewpoint of enhancing the light shielding property of visible light, the resin composition preferably contains the xanthene compound (b) having a maximum absorption wavelength in any of a range of 580 nm or more and 700 nm or less and a colorant (d-2) having a maximum absorption wavelength in any of a range of 490 nm or more and less than 580 nm at 350 to 800 nm described below, and further contains a colorant (d-1) having a maximum absorption wavelength in a range of 400 nm or more and less than 490 nm at 350 to 800 nm described below or a thermally coloring compound described below.

The xanthene compound (b) of the present invention can be produced in accordance with a known method for producing a xanthene compound, and is not particularly limited.

For example, a dichloride of sulfone fluorescein and a corresponding aromatic amine compound are heated and stirred in a solvent and cooled at room temperature, and this reaction solution is poured into a hydrochloric acid aqueous solution. Subsequently, the precipitate is collected by filtration, washed with water or hot water, and then dried to obtain a xanthene compound in which two nitrogen atoms are substituted with the same aryl group. In the case of producing a xanthene compound in which two nitrogen atoms are substituted with different aryl groups, the xanthene compound can be obtained by adding the corresponding half of the aromatic amine compound dropwise little by little into a solvent containing a dichloride of sulfone fluorescein, and after the reaction, adding the remaining aromatic amine compound dropwise.

Subsequently, a xanthene compound in which two nitrogen atoms are substituted with an aryl group and a corresponding aromatic halogen compound are heated and stirred in a solvent containing a copper catalyst and a base, and this reaction solution is filtered to remove insoluble matters, and then poured into a hydrochloric acid aqueous solution and stirred. Subsequently, the precipitate is collected by filtration, washed with water or hot water, and then dried to obtain a xanthene compound in which three or four nitrogen atoms are substituted with the same aryl group. In the case of a xanthene compound in which three nitrogen atoms are substituted with an aryl group, a xanthene compound in which four nitrogen atoms are substituted with an aryl group or a xanthene compound in which three nitrogen atoms are substituted with an aryl group and one nitrogen atom is substituted with an alkyl group can be obtained by performing the reaction in the same manner using a different aromatic halogen compound or aliphatic halogen compound.

<Alkali-Soluble Resin (a)>

The resin composition of the present invention contains the xanthene compound (b) of the present invention and an alkali-soluble resin (a). The alkali solubility refers to allowing the dissolution rate determined from a reduction in film thickness in the case of applying a solution of the resin dissolved in γ-butyrolactone onto a silicon wafer, forming a prebaked film of 10 μm±0.5 μm in film thickness by pre-baking for 4 minutes at 120° C., immersing the prebaked film in a 2.38 mass % tetramethylammonium hydroxide aqueous solution at 23±1° C. for 1 minute, and then subjecting the film to a rinse treatment with pure water, to be 50 nm/minute or more.

The alkali-soluble resin (a) has a hydroxyl group and/or an acidic group in the structural unit of the resin and/or the main chain terminal thereof in order to have alkali solubility. As the acidic group, the alkali-soluble resin (a) can have, for example, a carboxy group, a phenolic hydroxyl group, a sulfonic acid group, and the like.

Examples of the alkali-soluble resin (a) can include, but are not limited to, a polyimide, a polyimide precursor, a polybenzoxazole precursor, a polyamide-imide, a polyamide-imide precursor, a polyamide, a polymer of a radically polymerizable monomer having an acidic group, and a phenolic resin. The resin composition may contain two or more kinds of these resins.

Among them, since the development adhesion is high, heat resistance is excellent, and the amount of outgas at a high temperature is small, long-term reliability when the cured object is used in an organic EL display device, and thus the alkali-soluble resin (a) preferably includes one or more selected from the group consisting of a polyimide, a polyimide precursor, a polybenzoxazole, a polybenzoxazole precursor, a polyamide-imide, a polyamide-imide precursor, and a copolymer thereof, and more preferably includes a polyimide, a polyimide precursor, a polybenzoxazole precursor, or a copolymer thereof. From the viewpoint of further improving the sensitivity, a polyimide precursor or a polybenzoxazole precursor is still more preferable. The term “polyimide precursor” refers to a resin that is converted into a polyimide by heat treatment or chemical treatment. Examples of the polyimide precursor can include a polyamic acid and a polyamic acid ester. Here, the term “polybenzoxazole precursor” refers to a resin that is converted into polybenzoxazole by heat treatment or chemical treatment, and examples of the polybenzoxazole precursor can include polyhydroxyamide.

The polyimide precursor and the polybenzoxazole precursor describe above have a structural unit represented by the following Formula (3), and the polyimide has a structural unit represented by the following Formula (4). Two or more kinds of these may be contained, or a resin obtained by copolymerizing a structural unit represented by Formula (3) and a structural unit represented by Formula (4) may be contained.

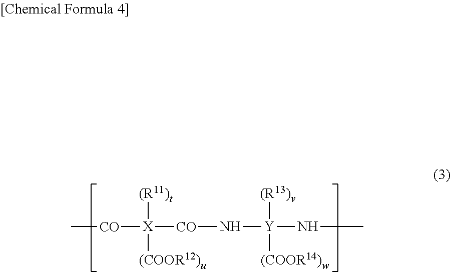

In Formula (3), X represents a divalent to octavalent organic group having 4 to 40 carbon atoms, and Y represents a divalent to undecavalent organic group having 6 to 40 carbon atoms. R¹¹ and R¹³ each independently represent a hydroxyl group or a sulfonic acid group. R¹² and R¹⁴ each independently represent a hydrogen atom or a monovalent hydrocarbon group having 1 to 20 carbon atoms. t, u, and w represent an integer of 0 to 3 and v represents an integer of 0 to 6. Provided that, t+u+v+w>0.

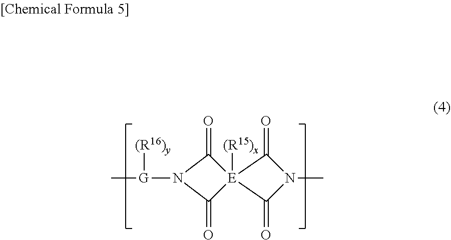

In Formula (4), E represents a tetravalent to decavalent organic group having 4 to 40 carbon atoms, and G represents a divalent to octavalent organic group having 6 to 40 carbon atoms. R¹⁵ and R¹⁶ each independently represent a carboxy group, a sulfonic acid group, or a hydroxyl group. x and y each independently represents an integer of 0 to 6. Provided that, x+y>0.

It is preferable that the polyimide, the polyimide precursor, the polybenzoxazole precursor, or a copolymer thereof has 5 to 100,000 of a structural unit represented by Formula (3) or (4). In addition to the structural unit represented by Formula (3) or (4), another structural unit may be contained. In this case, 50 mol % or more of the structural unit represented by Formula (3) or (4) based on the whole structural units is preferably contained.

In Formula (3), X(R¹¹)ₜ(COOR¹²)ᵤ represents a residue of an acid. X is a divalent to octavalent organic group having 4 to 40 carbon atoms, and preferably a divalent to octavalent organic group containing an aromatic ring or a cyclic aliphatic group.

Examples of the residue of the acid may include residues of dicarboxylic acids such as terephthalic acid, isophthalic acid, diphenyl ether dicarboxylic acid, bis(carboxyphenyl)hexafluoropropane, biphenyldicarboxylic acid, benzophenonedicarboxylic acid, and triphenyldicarboxylic acid, residues of tricarboxylic acids such as trimellitic acid, trimesic acid, diphenyl ether tricarboxylic acid, and biphenyltricarboxylic acid, and residues of tetracarboxylic acids such as aromatic tetracarboxylic acids such as pyromellitic acid, 3,3′,4,4′-biphenyltetracarboxylic acid, 2,3,3′,4′-biphenyltetracarboxylic acid, 2,2′,3,3′-biphenyltetracarboxylic acid, 3,3′,4,4′-benzophenonetetracarboxylic acid, 2,2′,3,3′-benzophenonetetracarboxylic acid, 2,2-bis(3,4-dicarboxyphenyl)hexafluoropropane, 2,2-bis(2,3-dicarboxyphenyl)hexafluoropropane, 1,1-bis(3,4-dicarboxyphenyl)ethane, 1,1-bis(2,3-dicarboxyphenyl)ethane, bis(3,4-dicarboxyphenyl)methane, bis(2,3-dicarboxyphenyl)methane, bis(3,4-dicarboxyphenyl) ether, 1,2,5,6-naphthalenetetracarboxylic acid, 2,3,6,7-naphthalenetetracarboxylic acid, 2,3,5,6-pyridinetetracarboxylic acid, 3,4,9,10-perylenetetracarboxylic acid, aromatic tetracarboxylic acids having the structures presented below, aliphatic tetracarboxylic acids such as butanetetracarboxylic acid, and aliphatic tetracarboxylic acids having a cyclic aliphatic group such as 1,2,3,4-cyclopentanetetracarboxylic acid. X(R¹¹)ₜ(COOR¹²)ᵤ may two or more kinds of these residues.

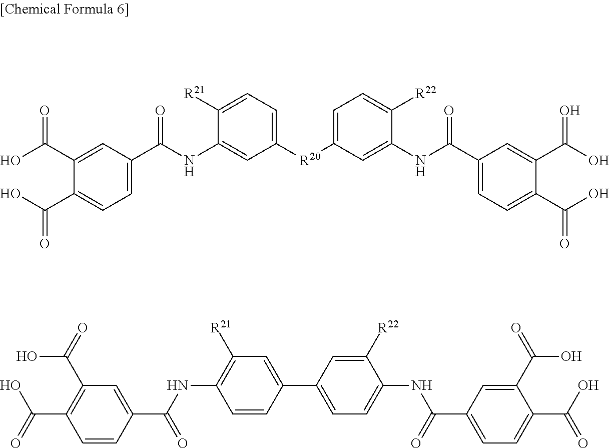

R²⁰ represents an oxygen atom, C (CF₃)₂, or C (CH₃)₂. R²¹ and R²² each independently represent a hydrogen atom or a hydroxyl group.

Among the residues of the acid, one or two carboxy groups correspond to (COOR¹²) in Formula (3) in the case of a residue of tricarboxylic acid or a tetracarboxylic acid.

In the above Formula (4), E(R¹⁵)ₓ represents of a residue of a dianhydride. E is a tetravalent to decavalent organic group having 4 to 40 carbon atoms, and preferably an organic group containing an aromatic ring or a cyclic aliphatic group.

Specific examples of the residue of the dianhydride include residues of aromatic tetracarboxylic dianhydrides such as pyromellitic dianhydride, 3,3′,4,4′-biphenyltetracarboxylic dianhydride, 2,3,3′,4′-biphenyltetracarboxylic dianhydride, 2,2′,3,3′-biphenyltetracarboxylic dianhydride, 3,3′,4,4′-benzophenone tetracarboxylic dianhydride, 2,2′,3,3′-benzophenone tetracarboxylic dianhydride, 2,2-bis(3,4-dicarboxyphenyl)propane dianhydride, 2,2-bis(2,3-dicarboxyphenyl)propane dianhydride, 1,1-bis(3,4-dicarboxyphenyl)ethane dianhydride, 1,1-bis(2,3-dicarboxyphenyl)ethane dianhydride, bis(3,4-dicarboxyphenyl)methane dianhydride, bis(2,3-dicarboxyphenyl)methane dianhydride, bis(3,4-dicarboxyphenyl)ether dianhydride, 1,2,5,6-naphthalenetetracarboxylic dianhydride, 9,9-bis(3,4-dicarboxyphenyl)fluorenic dianhydride, 9,9-bis{4-(3,4-dicarboxyphenoxy)phenyl}fluorenic dianhydride, 2,3,6,7-naphthalenetetracarboxylic dianhydride, 2,3,5,6-pyridinetetracarboxylic dianhydride, 3,4,9,10-perylenetetracarboxylic dianhydride, 2,2-bis(3,4-dicarboxyphenyl)hexafluoropropane dianhydride, and dianhydrides having the structures presented below, aliphatic tetracarboxylic dianhydrides such as butanetetracarboxylic dianhydride, and aliphatic tetracarboxylic dianhydrides containing a cyclic aliphatic group, such as 1,2,3,4-cyclopentanetetracarboxylic dianhydride. E(R¹⁵)ₓ may two or more kinds of these residues.

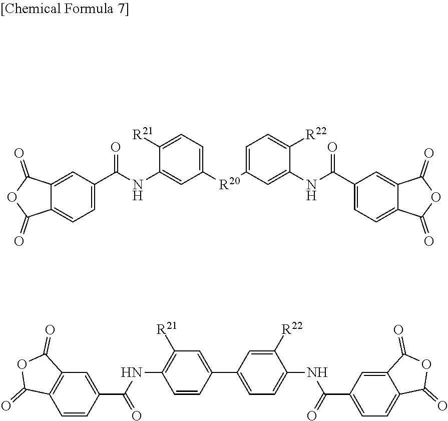

R²⁰ represents an oxygen atom, C (CF₃)₂, or C (CH₃)₂. R²¹ and R²² each independently represent a hydrogen atom or a hydroxyl group.

Y(R¹³)ᵥ(COOR¹⁴)_{w} in the above Formula (3) and G(R¹⁶)_{y} in the above Formula (4) each represent a residue of a diamine. Y is a divalent to undecavalent organic group having 6 to 40 carbon atoms, and preferably a divalent to undecavalent organic group containing an aromatic ring or a cyclic aliphatic group. G is a divalent to octavalent organic group having 6 to 40 carbon atoms, and preferably a divalent to octavalent organic group containing an aromatic ring or a cyclic aliphatic group.

Specific examples of the residue of the diamine can include residues of 3,4′-diaminodiphenyl ether, 4,4′-diaminodiphenyl ether, 3,4′-diaminodiphenylmethane, 4,4′-diaminodiphenylmethane, 1,4-bis(4-aminophenoxy)benzene, benzidine, m-phenylenediamine, p-phenylenediamine, 1,5-naphthalenediamine, 2,6-naphthalenediamine, bis(4-aminophenoxy)biphenyl, bis{4-(4-aminophenoxy)phenyl}ether, 1,4-bis(4-aminophenoxy)benzene, 2,2′-dimethyl-4,4′-diaminobiphenyl, 2,2′-diethyl-4,4′-diaminobiphenyl, 3,3′-dimethyl-4,4′-diaminobiphenyl, 3,3′-diethyl-4,4′-diaminobiphenyl, 2,2′,3,3′-tetramethyl-4,4′-diaminobiphenyl, 3,3′,4,4′-tetramethyl-4,4′-diaminobiphenyl, 2,2′-di(trifluoromethyl)-4,4′-diaminobiphenyl, 9,9-bis(4-aminophenyl)fluorene, 2,2′-bis(trifluoromethyl)-5,5′-dihydroxybenzidine, 3,5-diaminobenzoic acid, 3,4-diaminobenzoic acid, 2,5-diaminobenzoic acid, residues of aromatic diamines such as compounds obtained by substituting at least some of the hydrogen atoms of aromatic rings of these with an alkyl group or a halogen atom, residues of aliphatic diamines having a cyclic aliphatic group such as cyclohexyldiamine and methylenebiscyclohexylamine, and residues of diamines having the structures presented below. Y(R¹³)ᵥ(COOR¹⁴)_{w} and G(R¹⁶)_{y} may two or more kinds of these residues.

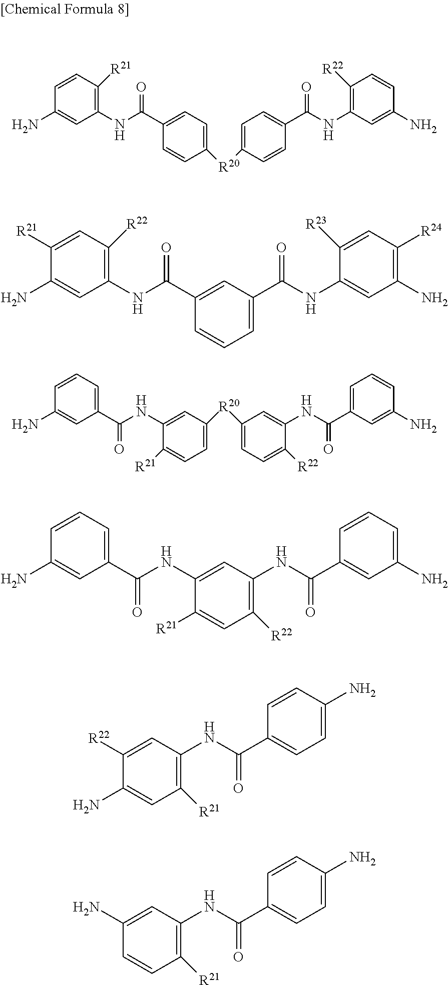

R²⁰ represents an oxygen atom, C (CF₃)₂, or C (CH₃)₂. R²¹ to R²⁴ each independently represent a hydrogen atom or a hydroxyl group.

The terminal of the alkali-soluble resin (a) may be sealed with a monoamine, an acid anhydride, an acid chloride, a monocarboxylic acid, or an active ester compound having a known acidic group.

The alkali-soluble resin (a) may be synthesized by a known method.

Examples of the method of producing a polyamic acid as a polyimide precursor include a method in which tetracarboxylic dianhydride and a diamine compound are reacted in a solvent at a low temperature.

Examples of the method of producing a polyamic acid ester as a polyimide precursor like a polyamic acid include, in addition to the above-described method in which a polyamic acid is reacted with an esterifying agent, a method in which a diester is obtained from tetracarboxylic dianhydride and an alcohol, and then the diester is reacted with an amine in a solvent in the presence of a condensing agent, and a method in which a diester is obtained from tetracarboxylic dianhydride and an alcohol, then the remaining dicarboxylic acid is converted into an acid chloride, and the acid chloride is reacted with an amine in a solvent. From the viewpoint of ease of synthesis, a step of reacting a polyamic acid with an esterifying agent is preferably included. The esterifying agent is not particularly limited, and a known method can be applied, but N,N-dimethylformamide dialkyl acetal is preferable because the obtained resin is easily purified.

Examples of a method for producing polyhydroxyamide which is polybenzoxazole precursor include a method in which a bisaminophenol compound and a dicarboxylic acid are subjected to a condensation reaction in a solvent. Specific examples thereof include a method in which a dehydration condensing agent such as dicyclohexylcarbodiimide (DCC) is reacted with an acid, and a bisaminophenol compound is added thereto, and a method in which a solution of dicarboxylic acid dichloride is added dropwise to a solution of a bisaminophenol compound to which a tertiary amine such as pyridine is added.

Examples of a method for producing polyimide include a method in which the polyamic acid or polyamic acid ester obtained by the above-described method is subjected to dehydration cyclization in a solvent. Examples of a method of dehydration cyclization include a chemical treatment using an acid, a base and the like and heat treatment.

Examples of a method for producing polybenzoxazole include a method in which the polyhydroxyamide obtained by the above-described method is subjected to dehydration cyclization in a solvent. Examples of a method of dehydration cyclization include a chemical treatment using an acid, a base and the like and heat treatment.

Examples of the polyamide-imide precursor include polymers of a tricarboxylic acid, a corresponding tricarboxylic anhydride, and a tricarboxylic anhydride halide with a diamine compound, and a polymer of trimellitic anhydride chloride with an aromatic diamine compound is preferable. Examples of a method for producing the polyamide-imide precursor include a method in which a tricarboxylic acid, a corresponding tricarboxylic anhydride, a tricarboxylic anhydride halide or the like is reacted with a diamine compound at a low temperature in a solvent.

Examples of a method for producing the polyamide-imide include a method in which trimellitic anhydride is reacted with an aromatic diisocyanate in a solvent and a method in which the polyamide-imide precursor obtained by the above-described method is subjected to dehydration cyclization in a solvent. Examples of a method of dehydration cyclization include a chemical treatment using an acid, a base and the like and heat treatment.

The polymerization solvent is not particularly limited, and examples thereof can include alkylene glycol monoalkyl ethers such as ethylene glycol monomethyl ether and propylene glycol monomethyl ether, alkyl acetates such as propyl acetate, butyl acetate, and isobutyl acetate, ketones such as methyl isobutyl ketone and methyl propyl ketone, alcohols such as butyl alcohol and isobutyl alcohol, ethyl lactate, butyl lactate, dipropylene glycol dimethyl ether, diethylene glycol dimethyl ether, diethylene glycol ethyl methyl ether, diethylene glycol diethyl ether, 3-methoxybutyl acetate, ethylene glycol monoethyl ether acetate, gamma butyrolactone, N-methyl-2-pyrrolidone, diacetone alcohol, N-cyclohexyl-2-pyrrolidone, N,N-dimethylformamide, N,N-dimethylacetamide, dimethyl sulfoxide, propylene glycol monomethyl ether acetate, N,N-dimethylisobutyramide, 3-methoxy-N,N-dimethylpropionamide, 3-butoxy-N,N-dimethylpropionamide, 1,3-dimethyl-2-imidazolidinone, N,N-dimethylpropyleneurea, delta valerolactone, 2-phenoxyethanol, 2-pyrrolidone, 2-methyl-1,3-propanediol, diethylene glycol butyl ether, triacetin, butyl benzoate, cyclohexylbenzene, bicyclohexyl, o-nitroanisole, diethylene glycol monobutyl ether, triethylene glycol monomethyl ether, N-(2-hydroxyethyl)-2-pyrrolidone, N,N-dimethylpropanamide, N,N-dimethylisobutyramide, N,N,N′,N′-tetramethylurea, and 3-methyl-2-oxazolidinone.

The content of the alkali-soluble resin (a) is preferably 40 mass % to 90 mass % in 100 mass % of solid contents of the resin composition. When the content of the alkali-soluble resin (a) is in this range, the light shielding property of the cured film can be enhanced while maintaining the heat resistance of the resin composition.

<Photosensitive Compound (c)>

The resin composition of the present invention may further contain a photosensitive compound (c).

When the resin composition of the present invention contains the photosensitive compound (c), the content of the photosensitive compound (c) is preferably 0.1 parts by mass or more, more preferably 1 part by mass or more, and still more preferably 10 parts by mass or more based on 100 parts by mass of the alkali-soluble resin (a) from the viewpoint of enhancing the sensitivity. On the other hand, the content thereof is preferably 100 parts by mass or less from the viewpoint of long-term reliability when the cured object of the present invention is used as a planarization layer and/or an insulation layer in an organic EL display device.

Examples of the photosensitive compound (c) can include a photo acid generator (c1) and a photo initiator (c2). The photo acid generator (c1) is a compound that generates an acid when irradiated with light, and the photo initiator (c2) is a compound that generates a radical by bond cleavage and/or reaction when exposed.

By the fact that the photo acid generator (c1) is included, a positive relief pattern can be obtained in which the part irradiated with light is dissolved because an acid is generated in the part irradiated with light to increase the solubility of the part in an alkali aqueous solution. Furthermore, by the fact that the photo acid generator (c1) and an epoxy compound or a thermal crosslinking agent described below are included, a negative relief pattern can be obtained in which the part irradiated with light is insolubilized because the acid generated in the part irradiated with light promotes the crosslinking reaction of the epoxy compound or the thermal crosslinking agent. By the fact that the photo initiator (c2) and a radically polymerizable compound described below are included, a negative relief pattern can be obtained in which the part irradiated with light is insolubilized because radical polymerization proceeds in the part irradiated with light. From the viewpoint that a fine pattern can be formed when the cured object of the present invention is used as a planarization layer and/or an insulation layer in an organic EL display device, it is preferable to contain the photo acid generator (c1) capable of obtaining a positive relief pattern as the photosensitive compound (c).

Examples of the photo acid generator (c1) can include quinone diazide compounds, sulfonium salts, phosphonium salts, diazonium salts, and iodonium salts. The resin composition of the present invention preferably contains two or more photo acid generators (c1), and when the resin composition contains two or more photo acid generators, a photosensitive resin composition having further high sensitivity can be obtained. From the viewpoint of the long-term reliability when the cured object of the present invention is used as a planarization layer and/or an insulation layer in an organic EL display device, the photo acid generator (c1) particularly preferably contains a quinone diazide compound.

Examples of the quinone diazide compound can include compounds in which a sulfonic acid of quinonediazide is bonded to a polyhydroxy compound to form an ester, compounds in which a sulfonic acid of quinonediazide is sulfonamide-bonded to a polyamino compound, and compounds in which a sulfonic acid of quinonediazide is ester-bonded and/or sulfonamide-bonded to a polyhydroxypolyamino compound.

As the quinone diazide, either a 5-naphthoquinone diazide sulfonyl group or a 4-naphthoquinone diazide sulfonyl group is preferably used. A naphthoquinone diazide sulfonyl ester compound having a 4-naphthoquinone diazide sulfonyl group and a 5-naphthoquinone diazide sulfonyl group in one molecule may be included, or a 4-naphthoquinone diazide sulfonyl ester compound and a 5-naphthoquinone diazide sulfonyl ester compound may be included. The 4-naphthoquinone diazide sulfonyl ester compound has absorption in the i-line region of a mercury lamp, and is suitable for i-line exposure. The 5-naphthoquinone diazide sulfonyl ester compound has an absorption in a region extending to the g-line region of a mercury lamp, and is suitable for g-line exposure.

It is preferable to select a 4-naphthoquinone diazide sulfonyl ester compound or a 5-naphthoquinone diazide sulfonyl ester compound according to the wavelength of light for exposure, but a 4-naphthoquinone diazide sulfonyl ester compound is preferably included from the viewpoint of enhancing the sensitivity.

The quinone diazide compound can be synthesized by an arbitrary esterification reaction from a compound having a phenolic hydroxyl group and a quinone diazide sulfonic acid compound. Use of these quinone diazide compounds leads to further improvement in the resolution, the sensitivity, and the residual film rate.

In the present invention, sulfonium salts, phosphonium salts, diazonium salts, and iodonium salts are preferable among the photo acid generators (c1) because these salts moderately stabilize the acid component generated through exposure. Among the salts, sulfonium salts are preferable. Furthermore, a sensitizer or the like may be included if necessary.

When the resin composition contents the photo acid generator (c1), the content of the photo acid generator (c1) is preferably 0.1 parts by mass or more, more preferably 10 parts by mass or more, and still more preferably 25 parts by mass or more based on 100 parts by mass of the alkali-soluble resin (a) from the viewpoint of enhancing the sensitivity. On the other hand, the content thereof is preferably 100 parts by mass or less from the viewpoint of long-term reliability when the cured object of the present invention is used as a planarization layer and/or an insulation layer in an organic EL display device.

Examples of the photo initiator (c2) can include benzyl ketal-based photo initiators, α-hydroxyketone-based photo initiators, α-aminoketone-based photo initiators, acylphosphine oxide-based photo initiators, oxime ester-based photo initiators, acridine-based photo initiators, titanocene-based photo initiators, benzophenone-based photo initiators, acetophenone-based photo initiators, aromatic ketoester-based photo initiators, and benzoic acid ester-based photo initiators. The resin composition of the present invention may contain two or more of the photo initiators (c2). From the viewpoint of further improving the sensitivity, the photo initiator (c2) still more preferably includes an α-aminoketone-based photo initiator, an acylphosphine oxide-based photo initiator, or an oxime ester-based photo initiator.

Examples of the α-aminoketone-based photo initiator can include 2-methyl-1-[4-(methylthio)phenyl]-2-morpholinopropane-1-one, 2-benzyl-2-dimethylamino-1-(4-morpholinophenyl)-butan-1-one, 2-dimethylamino-2-(4-methylbenzyl)-1-(4-morpholinophenyl)-butan-1-one, and 3,6-bis(2-methyl-2-morpholinopropionyl)-9-octyl-9H-carbazol e.

Examples of the acylphosphine oxide-based photo initiator can include 2,4,6-trimethylbenzoyl-diphenylphosphine oxide, bis(2,4,6-trimethylbenzoyl)-phenylphosphine oxide, and bis(2,6-dimethoxybenzoyl)-(2,4,4-trimethylpentyl)phosphine oxide.

Examples of the oxime ester-based photo initiator can include 1-phenylpropane-1,2-dione-2-(0-ethoxycarbonyl)oxime, 1-phenylbutane-1,2-dione-2-(0-methoxycarbonyl)oxime, 1,3-diphenylpropane-1,2,3-trione-2-(0-ethoxycarbonyl)oxime, 1-[4-(phenylthio)phenyl]octane-1,2-dione-2-(0-benzoyl)oxime, 1-[4-[4-(carboxyphenyl)thio]phenyl]propane-1,2-dione-2-(O-a cetyl)oxime, 1-[9-ethyl-6-(2-methylbenzoyl)-9H-carbazole-3-yl]ethanone-1-(O-acetyl)oxime, 1-[9-ethyl-6-[2-methyl-4-[1-(2,2-dimethyl-1,3-dioxolan-4-yl)methyloxy]benzoyl]-9H-carbazol-3-yl]ethanone-1-(O-acetyl)oxime, and 1-(9-ethyl-6-nitro-9H-carbazole-3-yl)-1-[2-methyl-4-(1-meth oxypropane-2-yloxy)phenyl]methanone-1-(0-acetyl)oxime.

In the present invention, when the photo initiator (c2) is contained, the content of the photo initiator (c2) is preferably 0.1 parts by mass or more, more preferably 1 part by mass or more, and still more preferably 10 parts by mass or more based on 100 parts by mass of the total of the alkali-soluble resin (a) and the radically polymerizable compound described below, from the viewpoint of enhancing the sensitivity. The content is preferably 50 parts by mass or less from the viewpoint of further improving the resolution and reducing the taper angle.

<Colorant (d)>

The resin composition of the present invention may contain a colorant (d) other than the xanthene compound (b) When the colorant (d) is contained, a light shielding property can be imparted for shielding light having a wavelength absorbed by the colorant (d) from light transmitted through the film of the resin composition or light reflected from the film of the resin composition. When the cured object of the present invention described below is used as a planarization layer and/or an insulation layer in an organic EL display device, if the light shielding property is imparted, it is possible to prevent deterioration, malfunction, leakage current, and the like due to intrusion of light into the TFT. Furthermore, external light reflection from the wiring and the TFT can be suppressed, and the contrast between the light-emitting area and the non-light-emitting area can be improved.

As the colorant (d), a dye (d1) and/or a pigment (d2) is preferably used. The colorant (d) preferably includes at least one dye or organic pigment, and for example, preferably includes one dye or organic pigment, two or more dyes or organic pigments, or one or more dyes and one or more pigments.

The colorant (d) in the present invention is preferably a dye (d1) from the viewpoint of solubility in a solvent. From the viewpoint of enhancing the sensitivity and reducing the residues, the dye (d1) is preferably an ionic dye (d10) forming an ion pair of organic ions (hereinafter, may referred to as the ionic dye (d10)). The pigment (d2) is preferable from the viewpoint that discoloration of the colorant in the step of subjecting the resin composition of the present invention described below to heat treatment.

The resin composition of the present invention preferably contains a colorant (d-2) having a maximum absorption wavelength in any of a range of 490 nm or more and less than 580 nm at 350 to 800 nm, and specifically preferably contains a dye (d1-2) having a maximum absorption wavelength in any of a range of 490 nm or more and less than 580 nm at 350 to 800 nm and/or a pigment (d2-2) having a maximum absorption wavelength in any of a range of 490 nm or more and less than 580 nm at 350 to 800 nm. Hereinafter, the above-described dye and pigment are sometimes simply referred to as a component (d-2), a component (d1-2), and a component (d2-2), respectively.

In the present invention, the component (d1-2) preferably includes a dye that is soluble in an organic solvent that dissolves the alkali-soluble resin (a) and compatible with a resin, or a dye that has high heat resistance and high light resistance, from the viewpoint of storage stability and discoloration at the time of curing or light irradiation. The component (d1-2) has a maximum absorption wavelength in any of a range of 490 nm or more and less than 580 nm at 350 to 800 nm, so that examples thereof include red dyes and purple dyes. Examples of the dye can include oil-soluble dyes, disperse dyes, reactive dyes, acidic dyes, and direct dyes.

Examples of the skeleton structure of the dye include anthraquinone-based, azo-based, phthalocyanine-based, methine-based, oxazine-based, quinoline-based, triarylmethane-based, and xanthene-based structures, but are not limited thereto. Among the skeleton structures, anthraquinone-based, azo-based, methine-based, triarylmethane-based, and xanthene-based structures are preferable from the viewpoint of solubility in an organic solvent and heat resistance. From the viewpoint of processability when the xanthene compound (b) of the present invention is formed into a resin composition, a xanthene-based structure is still more preferable. These dyes may be used singly or as a metal-containing complex salt system. Specifically, dyes having a maximum absorption wavelength in any of a range of 490 nm or more and less than 580 nm at 350 to 800 nm are available among Sumilan and Lanyl dyes (manufactured by Sumitomo Chemical Industry Company Limited), Orasol, Oracet, Filamid, and Irgasperse dyes (manufactured by Ciba Specialty Chemicals Inc.), Zapon, Neozapon, Neptune, and Acidol dyes (manufactured by BASF SE), Kayaset and Kayakalan dyes (manufactured by Nippon Kayaku Co., Ltd.), a Valifast Colors dye (manufactured by ORIENT CHEMICAL INDUSTRIES CO., LTD.), Savinyl, Sandoplast, Polysynthren, and Lanasyn dyes (manufactured by Clariant (Japan) K.K.), an Aizen Spilon dye (manufactured by Hodogaya Chemical Co., Ltd.), functional dyes (manufactured by Yamada Chemical Co., Ltd.), a Plast Color dye and an Oil Color dye (manufactured by ARIMOTO CHEMICAL Co., Ltd.), and the like, but the dyes are not limited thereto. These dyes are used singly or in combination.

In the present invention, the component (d-2) is preferably a pigment having high heat resistance and high light resistance from the viewpoint of discoloration at the time of curing or light irradiation.

Specific examples of the organic pigment are indicated by color index (CI) numbers. Examples of the red pigment include Pigment Red 48:1, 122, 168, 177, 202, 206, 207, 209, 224, 242, and 254. Examples of the violet pigment include Pigment Violet 19, 23, 29, 32, 33, 36, 37, and 38. A pigment other than these pigments can also be contained.

The content of the component (d-2) is preferably 0.1 to 300 parts by mass, more preferably 0.2 to 200 parts by mass, and particularly preferably 1 to 200 parts by mass based on 100 parts by mass of the alkali-soluble resin (a). When the content of the component (d-2) is 0.1 parts by mass or more, light having a corresponding wavelength can be absorbed. When the content is 300 parts by mass or less, light having a corresponding wavelength can be absorbed while the adhesion strength between the photosensitive colored resin film and the substrate, and the heat resistance and the mechanical characteristic of the heat-treated film are maintained.

In the present invention, an organic pigment used as the component (d2-2) may be contained that is subjected to surface treatment such as rosin treatment, acidic group treatment, or basic group treatment if necessary. The organic pigment can be contained together with a dispersant in some cases. Examples of the dispersant can include cation-based, anion-based, nonionic, amphoteric, silicone-based, and fluorine-based surfactants.

In the resin composition of the present invention, the colorant (d) may include a colorant (d-1) having a maximum absorption wavelength in any of a range of 400 nm or more and less than 490 nm at 350 to 800 nm, and specifically may include a dye (d1-1) having a maximum absorption wavelength in any of a range of 400 nm or more and less than 490 nm at 350 to 800 nm and/or a pigment (d2-1) having a maximum absorption wavelength in any of a range of 400 nm or more and less than 490 nm at 350 to 800 nm. Hereinafter, the above-described dye and pigment are sometimes simply referred to as a component (d-1), a component (d1-1), and a component (d2-1), respectively.

In the present invention, the dye (d1-1) used as the component (d-1) is preferably a dye that is soluble in an organic solvent that dissolves the alkali-soluble resin (a) and compatible with a resin, and has high heat resistance and high light resistance, from the viewpoint of storage stability and discoloration at the time of curing or light irradiation. The component (d1-1) has a maximum absorption at a wavelength in the range of 400 nm or more and less than 490 nm, so that examples thereof include yellow dyes and orange dyes. Examples of the dye include oil-soluble dyes, disperse dyes, reactive dyes, acidic dyes, and direct dyes.

Examples of the skeleton structure of the dye include anthraquinone-based, azo-based, phthalocyanine-based, methine-based, oxazine-based, quinoline-based, triarylmethane-based, and xanthene-based structures, but are not limited thereto. Among the skeleton structures, anthraquinone-based, azo-based, methine-based, triarylmethane-based, and xanthene-based structures are preferable from the viewpoint of solubility in an organic solvent and heat resistance. These dyes may be used singly or as a metal-containing complex salt system. Specifically, dyes having a maximum absorption wavelength in any of a range of 400 nm or more and less than 490 nm at 350 to 800 nm are available among Sumilan and Lanyl dyes (manufactured by Sumitomo Chemical Industry Company Limited), Orasol, Oracet, Filamid, and Irgasperse dyes (manufactured by Ciba Specialty Chemicals Inc.) Zapon, Neozapon, Neptune, and Acidol dyes (manufactured by BASF SE), Kayaset and Kayakalan dyes (manufactured by Nippon Kayaku Co., Ltd.), a Valifast Colors dye (manufactured by ORIENT CHEMICAL INDUSTRIES CO., LTD.), Savinyl, Sandoplast, Polysynthren, and Lanasyn dyes (manufactured by Clariant (Japan) K.K.), an Aizen Spilon dye (manufactured by Hodogaya Chemical Co., Ltd.), functional dyes (manufactured by Yamada Chemical Co., Ltd.), a Plast Color dye and an Oil Color dye (manufactured by ARIMOTO CHEMICAL Co., Ltd.), and the like, but the dyes are not limited thereto. These dyes are used singly or in combination.

In the present invention, the pigment (d2-1) used as the component (d-1) is preferably a pigment having high heat resistance and high light resistance from the viewpoint of discoloration at the time of curing or light irradiation.

Specific examples of the organic pigment are indicated by color index (CI) numbers. Examples of the yellow pigment include Pigment Yellow 83, 117, 129, 138, 139, 150, and 180. Examples of the orange pigment include Pigment Orange 38, 43, 64, 71, and 72. A pigment other than these pigments can also be contained.

The content of the component (d-1) is preferably 0.1 to 300 parts by mass, more preferably 0.2 to 200 parts by mass, and particularly preferably 1 to 200 parts by mass based on 100 parts by mass of the alkali-soluble resin (a). When the content of the component (d-1) is 0.1 parts by mass or more, light having a corresponding wavelength can be absorbed. When the content is 300 parts by mass or less, light having a corresponding wavelength can be absorbed while the adhesion strength between the photosensitive colored resin film and the substrate, and the heat resistance and the mechanical characteristic of the heat-treated film are maintained.

In the present invention, as the component (d2-1), an organic pigment may be used that is subjected to surface treatment such as rosin treatment, acidic group treatment, or basic group treatment if necessary. The organic pigment can be used together with a dispersant in some cases. Examples of the dispersant include cation-based, anion-based, nonionic, amphoteric, silicone-based, and fluorine-based surfactants.

In the resin composition of the present invention, the colorant (d) may include a colorant (d-3) having a maximum absorption wavelength in any of a range of 580 nm or more and 800 nm or less at 350 to 800 nm, and specifically may include a dye (d1-3) having a maximum absorption wavelength in any of a range of 580 nm or more and 800 nm or less at 350 to 800 nm and/or a pigment (d2-3) having a maximum absorption wavelength in any of a range of 580 nm or more and 800 nm or less at 350 to 800 nm. Hereinafter, the above-described dye and pigment are sometimes simply referred to as a component (d-3), a component (d1-3), and a component (d2-3), respectively.

In the present invention, the dye (d1-3) used as the component (d-3) is preferably a dye that is soluble in an organic solvent that dissolves the alkali-soluble resin (a) and compatible with a resin, and has high heat resistance and high light resistance, from the viewpoint of storage stability and discoloration at the time of curing or light irradiation. The component (d1-3) has a maximum absorption wavelength in any of a range of 580 nm or more and 800 nm or less at 350 to 800 nm, so that examples thereof include blue dyes and green dyes.

Examples of the dye include oil-soluble dyes, disperse dyes, reactive dyes, acidic dyes, and direct dyes.

Examples of the skeleton structure of the dye include anthraquinone-based, azo-based, phthalocyanine-based, methine-based, oxazine-based, quinoline-based, and triarylmethane-based structures, but are not limited thereto. Among the skeleton structures, anthraquinone-based, azo-based, and methine-based, triarylmethane-based structures are preferable from the viewpoint of solubility in an organic solvent and heat resistance. These dyes may be used singly or as a metal-containing complex salt system. Specifically, dyes having a maximum absorption wavelength in any of a range of 580 nm or more and 800 nm or less at 350 to 800 nm are available among Sumilan and Lanyl dyes (manufactured by Sumitomo Chemical Industry Company Limited), Orasol, Oracet, Filamid, and Irgasperse dyes (manufactured by Ciba Specialty Chemicals Inc.), Zapon, Neozapon, Neptune, and Acidol dyes (manufactured by BASF SE), Kayaset and Kayakalan dyes (manufactured by Nippon Kayaku Co., Ltd.), a Valifast Colors dye (manufactured by ORIENT CHEMICAL INDUSTRIES CO., LTD.), Savinyl, Sandoplast, Polysynthren, and Lanasyn dyes (manufactured by Clariant (Japan) K.K.), an Aizen Spilon dye (manufactured by Hodogaya Chemical Co., Ltd.), functional dyes (manufactured by Yamada Chemical Co., Ltd.), a Plast Color dye and an Oil Color dye (manufactured by ARIMOTO CHEMICAL Co., Ltd.), and the like, but the dyes are not limited thereto. These dyes are used singly or in combination.

In the present invention, the pigment (d2-3) used as the component (d-3) is preferably a pigment having high heat resistance and high light resistance from the viewpoint of discoloration at the time of curing or light irradiation.

Specific examples of the organic pigment are indicated by color index (CI) numbers. Examples of the blue pigment include Pigment Blue 15 (15:3, 15:4, 15:6, and the like), 21, 22, 60, and 64. Examples of the green pigment include Pigment Green 7, 10, 36, 47, and 58. A pigment other than these pigments can also be contained.

The content of the component (d-3) is preferably 0.1 to 300 parts by mass, more preferably 0.2 to 200 parts by mass, and particularly preferably 1 to 200 parts by mass based on 100 parts by mass of the alkali-soluble resin (a). When the content of the component (d-3) is 0.1 parts by mass or more, light having a corresponding wavelength can be absorbed. When the content is 300 parts by mass or less, light having a corresponding wavelength can be absorbed while the adhesion strength between the photosensitive colored resin film and the substrate, and the heat resistance and the mechanical characteristic of the heat-treated film are maintained.

In the present invention, as the component (d2-3), an organic pigment may be used that is subjected to surface treatment such as rosin treatment, acidic group treatment, or basic group treatment if necessary. The organic pigment can be used together with a dispersant in some cases. Examples of the dispersant include cation-based, anion-based, nonionic, amphoteric, silicone-based, and fluorine-based surfactants.

In the present invention, by using the xanthene compound (b), the component (d-2), the component (d-1) and/or a thermally coloring compound described below, and if necessary, the component (d-3) in combination, the visible light transmittance of the cured object can be lowered to give a black color. An optical density (hereinafter, may be referred to as OD value) per film thickness of 1 μm of the cured object obtained by curing the resin composition containing the xanthene compound (b) of the present invention is preferably 0.5 or more and more preferably 0.7 or more in OD value. When the OD value is within the above range, the light shielding property can be improved by the cured object, so that in display devices such as organic EL display devices or liquid crystal display devices, it becomes possible to reduce visualization of electrode wirings or reduce external light reflection. Therefore, contrast in image display can be improved. From the viewpoint that the sensitivity upon exposure to light can be improved when a resin composition containing a photosensitive compound described below is formed, the OD value is preferably 1.5 or less.

<Ionic Dye (d10) and Organic Anion>

It is preferable that the resin composition of the present invention contains a xanthene compound (b1) in which n is 1 and Z is an organic anion in Formula (1) (hereinafter, may referred to as the xanthene compound (b1)) and an ionic dye (d10) forming an ion pair of organic ions, and the organic anions are one kind. However, the ionic dye forming an ion pair of organic ions represents an ionic dye including individual organic anions and organic cations, and a compound that has an anion moiety and a cation moiety as a simple substance and is charge neutral as a whole, such as a xanthene compound in which n is 0 in Formula (1), is not counted as an organic anion. The fact that the organic anions are one kind means that the organic anion in the xanthene compound (b1) and the organic anion constituting the ionic dye (d10) are the same. When the resin composition of the present invention contains the xanthene compound (b1) and the ionic dye (d10) and the organic anion moieties are different from each other, the organic anions contained in the resin composition are two or more kinds. In this case, the presence of a plurality of organic anions and organic cations in the resin composition causes a problem that ion exchange between ionic dyes increases foreign matters during frozen storage, leading to deterioration of storage stability. When the xanthene compound (b1) and the ionic dye (d10) are contained, the organic anions contained in the resin composition of the present invention are one kind, thereby improving the storage stability during frozen storage. This is presumed to be because since the organic anion species for the xanthene compound (b1) and the ion dye (d10) were limited, ion exchange between ionic dyes was suppressed in the resin composition even when the organic cation moieties were different from each other.

The ionic dye (d10) forming an ion pair of organic ions in the present invention refers to a salt forming compound having an organic anion moiety of an acidic dye and an organic cation moiety of a non-dye, a salt forming compound including an organic cation moiety of a basic dye and an organic anion moiety of a non-dye, or a salt forming compound including an organic anion moiety of an acidic dye and an organic cation moiety of a basic dye.

The salt forming compound including an organic cation moiety of a basic dye and an organic anion moiety of a non-dye can be produced by using the basic dye as a raw material and exchanging the counter anion with the organic anion of the non-dye by a known method. The salt forming compound having an organic anion moiety of an acidic dye and an organic cation moiety of a non-dye can be produced by using the acidic dye as a raw material and exchanging the counter cation with the organic cation of the non-dye by a known method. The salt forming compound including an organic anion moiety of an acidic dye and an organic cation moiety of a basic dye can be produced by using the acidic dye and the basic dye as raw materials and exchanging the counter ions of the acidic dye and the basic dye by a known method.

The acidic dye as a raw material of the ionic dye (d10) is an anionic water-soluble dye which is a compound having an acidic substituent such as a sulfo group or a carboxy group in the molecule of the dye or a salt thereof. The acidic dye includes those having an acidic substituent such as a sulfo group or a carboxy group and classified as a direct dye.

Examples of the acidic dye include azo-based acidic dyes such as C.I. Acid Yellow 1, 17, 18, 23, 25, 36, 38, 42, 44, 54, 59, 72, 78, and 151; C.I. Acid Orange 7, 10, 12, 19, 20, 22, 28, 30, 52, 56, 74, and 127; C.I. Acid Red 1, 3, 4, 6, 8, 11, 12, 14, 18, 26, 27, 33, 37, 53, 57, 88, 106, 108, 111, 114, 131, 137, 138, 151, 154, 158, 159, 173, 184, 186, 215, 257, 266, 296, and 337; C.I. Acid Brown 2, 4, 13, and 248; C.I. Acid Violet 11, 56, and 58; and C.I. Acid Blue 92, 102, 113, and 117; quinoline-based acidic dyes such as C.I. Acid Yellow 2, 3, and 5; xanthene-based acidic dyes such as C.I. Acid Red 50, 51, 52, 87, 91, 92, 93, 94, and 289; anthraquinone-based acidic dyes such as C.I. Acid Red 82 and 92; C.I. Acid Violet 41, 42, and 43; C.I. Acid Blue 14, 23, 25, 27, 40, 45, 78, 80, 127:1, 129, 145, 167, and 230; and C.I. Acid Green 25 and 27; triarylmethane-based acidic dyes such as C.I. Acid Violet 49; C.I. Acid Blue 7, 9, 22, 83, and 90; C.I. Acid Green 9 and 50; and C.I. Food Green 3; phthalocyanine-based acidic dyes such as C.I. Acid Blue 249; and indigoid-based acidic dyes such as C.I. Acid Blue 74. Among them, the acid dye preferably includes xanthene-based acidic dyes from the viewpoint of high heat resistance. The xanthene-based acidic dyes more preferably include a rhodamine-based acidic dyes such as C.I. Acid Red 50, 52, and 289.

Examples of the organic cation moiety of the non-dye as a raw material of the ionic dye (d10) include ammonium ions [N(R)₄]⁺, phosphonium ions [P(R)₄]⁺, iminium ions [(R)₂—N═C(R)₂]⁺, arsonium ions [As(R)₄]⁺, stibonium ions [Sb (R)₄]⁺, oxonium ions [O(R)₃]⁺, sulfonium ions [S(R)₃]⁺, selenonium ions [Se(R)₃]⁺, stannonium ions [Sn(R)₃]⁺, iodonium ions [I(R)₂]⁺, and diazonium ions [R—N⁺═N]. From the viewpoint of insulation properties when the cured object formed of the resin composition of the present invention is applied, ammonium ions [N(R)₄]⁺, phosphonium ions [P(R)₄]⁺, and iminium ions [(R)₂—N═C(R)₂]⁺ are preferable. R in the ionic formula is each independently a hydrocarbon group having 1 to 20 carbon atoms which may have a substituent and may have a heteroatom in the carbon chain. From the viewpoint of improving the sensitivity by increasing the ratio of the coloring component per molecule and decreasing the addition amount of the ionic dye, the molecular weight of the organic cation moiety of the non-dye is preferably 1000 or less, preferably 700 or less, and still more preferably 300 or less. The lower limit of the molecular weight of the organic cation moiety of the non-dye is not particularly limited, and is preferably 1 or more and still more preferably 100 or more.

The basic dye as a raw material of the ionic dye (d10) is a compound having a basic group, such as an amino group or an imino group, in the molecule or a salt thereof, and is a dye that becomes a cation in an aqueous solution.

Examples of the basic dye include azo-based basic dyes such as C.I. Basic Red 17, 22, 23, 25, 29, 30, 38, 39, 46, 46:1, and 82; C.I. Basic Orange 2, 24, and 25; C.I. Basic Violet 18; C.I. Basic Yellow 15, 24, 25, 32, 36, 41, 73, and 80; C.I. Basic Brown 1; and C.I. Basic Blue 41, 54, 64, 66, 67, and 129; xanthene-based basic dyes such as C.I. Basic Red 1 and 2; and C.I. Basic Violet 10 and 11; methine-based basic dyes such as C.I. Basic Yellow 11, 13, 21, 23, and 28; C.I. Basic Orange 21; C.I. Basic Red 13 and 14; and C.I. Basic Violet 16 and 39; anthraquinone-based basic dyes such as C.I. Basic Blue 22, 35, 45, and 47; triarylmethane-based basic dyes such as C.I. Basic Violet 1, 2, 3, 4, 13, 14, and 23; C.I. Basic Blue 1, 5, 7, 8, 11, 15, 18, 21, 24, and 26; and C.I. Basic Green 1 and 4, and xanthene-based basic dyes having the structures presented below.

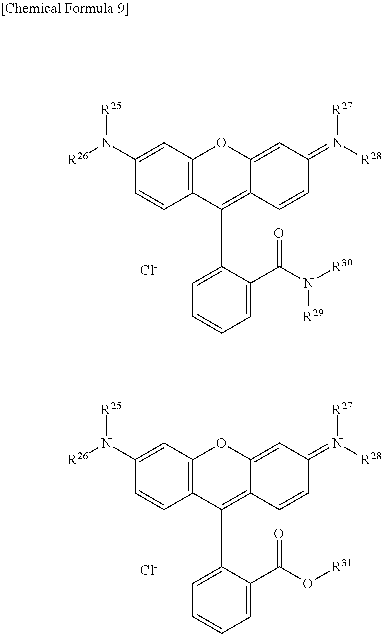

R²⁵, R²⁷, and R²⁹ to R³¹ each independently represent a hydrogen atom, an alkyl group having 1 to 10 carbon atoms, or an aryl group having 6 to 10 carbon atoms which may have a substituent, and R²⁶ and R²⁸ each independently represent a hydrogen atom or an alkyl group having 1 to 10 carbon atoms.

Among them, the basic dye preferably includes xanthene-based basic dyes and triarylmethane-based basic dyes from the viewpoint of increasing the blackness of the cured film, and preferably includes xanthene-based acidic dyes from the viewpoint of high heat resistance.

Examples of the organic anion moiety of the non-dye as a raw material of the ionic dye (d10) include, in addition to aliphatic or aromatic sulfonate ions and aliphatic or aromatic carboxylate ions, sulfonimide anions [(RSO₂)₂N]⁻ and borate anions (BR₄)⁻. From the viewpoint of suppressing deterioration of an electrode or a light-emitting layer in an organic EL display device when the cured object formed of the resin composition of the present invention is applied, the anionic compound is preferably an aliphatic or aromatic sulfonate ion or an aliphatic or aromatic carboxylate ion. From the viewpoint of enhancing the sensitivity and reducing the residues, an aliphatic or aromatic sulfonate ion is preferable. R in the ionic formula is each independently a hydrocarbon group having 1 to 20 carbon atoms which may have a substituent and may have a heteroatom in the carbon chain. From the viewpoint of improving the sensitivity by increasing the ratio of the coloring component per molecule and decreasing the addition amount of the ionic dye, the molecular weight of the organic anion moiety of the non-dye is preferably 1000 or less, preferably 700 or less, and still more preferably 300 or less. The lower limit of the molecular weight of the anion moiety of the non-dye is not particularly limited, and is preferably 1 or more and still more preferably 100 or more.

From the viewpoint of high heat resistance, the organic anion moiety and/or the organic cation moiety of the ionic dye (d10) preferably has a xanthene skeleton. Examples of the organic anion having a xanthene skeleton include the xanthene-based acidic dyes described above, and examples of the organic cation having a xanthene skeleton include the xanthene-based basic dyes described above.

The ionic dye (d10) preferably has an acidic group from the viewpoint of enhancing alkali solubility during development and improving the sensitivity. As the acidic group, the ionic dye (d10) can have, for example, a carboxy group, a phenolic hydroxyl group, a sulfonic acid group, a sulfonate group, or the like, and a sulfonic acid group or a sulfonate group is particularly preferable.

When the ionic dye (d10) is used in combination with the xanthene compound (b), from the viewpoint of enhancing the light shielding property of visible light, the ionic dye (d10) preferably contains a colorant (d10-2) having a maximum absorption wavelength in any of a range of 490 nm or more and less than 580 nm at 350 to 800 nm.

A salt forming compound obtained by ion exchange of the acidic dye or the basic dye can be produced by a known method. For example, when an aqueous solution of an acidic dye and an aqueous solution of a basic dye are prepared and both are slowly mixed under stirring, a salt forming compound including an organic anion moiety of the acidic dye and an organic cation moiety of the basic dye is produced as a precipitate. The salt forming compound can be obtained by collecting the precipitate by filtration. The obtained salt forming compound is preferably dried at about 60 to 70° C.

The total content of the ionic dye (d10) contained in the resin composition of the present invention is preferably 0.1 parts by mass or more and 300 parts by mass or less, more preferably 0.2 parts by mass or more and 200 parts by mass or less, and particularly preferably 1 part by mass or more and 200 parts by mass or less based on 100 parts by mass of the alkali-soluble resin (a). When the content of the ionic dye (b) is 0.1 parts by mass or more, light having a corresponding wavelength can be absorbed. When the content is 300 parts by mass or less, light having a corresponding wavelength can be absorbed while the adhesion strength between the photosensitive colored resin film and the substrate, and the heat resistance and the mechanical characteristic of the heat-treated film are maintained.

### <Thermally Coloring Compound>

The resin composition of the present invention may contain a thermally coloring compound. The thermally coloring compound develops a color by heat treatment and has a maximum absorption at 350 nm or more and 700 nm or less, and more preferably develops a color by heat treatment and has a maximum absorption at 350 nm or more and 500 nm or less.

In the present invention, the thermally coloring compound preferably develops a color at a temperature higher than 120° C., and more preferably develops a color at a temperature higher than 180° C. The higher the temperature at which the thermally coloring compound develops a color is, the more excellent the heat resistance under a high-temperature condition is, and the more excellent the light resistance is so that the less discoloration is caused by long-time irradiation with ultraviolet light or visible light.

In the present invention, the thermally coloring compound may be a general heat-sensitive dye or pressure-sensitive dye, or may be another compound. Examples of the thermally coloring compound can include a compound that develops a color by changing its chemical structure or charge state due to the action of an acidic group coexisting in the system during heat treatment, and a compound that develops a color by a thermal oxidation reaction or the like due to oxygen existing in the air. The thermally coloring compound of the present invention does not have a maximum absorption in any of a range of 350 nm or more and 700 nm or less before the heat treatment, and thus is different from the colorant (d). For example, a thermally coloring compound having a triarylmethane skeleton develops a color when hydrogen of a methine group is eliminated by heat treatment and one aryl group becomes a quinone structure. On the other hand, the coloring material (d) having a triarylmethane skeleton has a quinone structure before the heat treatment, and thus is different from the thermally coloring compound of the present invention.

Examples of the skeleton structure of the thermally coloring compound can include a triarylmethane skeleton, a diarylmethane skeleton, a fluoran skeleton, a bislactone skeleton, a phthalide skeleton, a xanthene skeleton, a rhodamine lactam skeleton, a fluorene skeleton, a phenothiazine skeleton, a phenoxazine skeleton, and a spiropyran skeleton. Among the skeletons, a triarylmethane skeleton is preferable because with a triarylmethane skeleton, the thermally coloring temperature is high, and the heat resistance is excellent.

Specific examples of the triarylmethane skeleton can include 2,4′,4″-methylidyne trisphenol, 4,4′,4″-methylidyne trisphenol, 4,4′-[(4-hydroxyphenyl)methylene]bis(benzenamine), 4,4′-[(4-aminophenyl)methylene]bisphenol, 4,4′-[(4-aminophenyl)methylene]bis[3,5-dimethylphenol], 4,4′-[(2-hydroxyphenyl)methylene]bis[2,3,6-trimethylphenol], 4-[bis(4-hydroxyphenyl)methyl]-2-methoxyphenol, 4,4′-[(2-hydroxyphenyl)methylene]bis[2-methylphenol], 4,4′-[(4-hydroxyphenyl)methylene]bis[2-methylphenol], 4-[bis(4-hydroxyphenyl)methyl]-2-ethoxyphenol, 4,4′-[(4-hydroxyphenyl)methylene]bis[2,6-dimethylphenol], 2,2′-[(4-hydroxyphenyl)methylene]bis[3,5-dimethylphenol], 4,4′-[(4-hydroxy-3-methoxyphenyl)methylene]bis[2,6-dimethyl phenol], 2,2′-[(2-hydroxyphenyl)methylene]bis[2,3,5-trimethylphenol], 4,4′-[(4-hydroxyphenyl)methylene]bis[2,3,6-trimethylphenol], 4,4′-[(2-hydroxyphenyl)methylene]bis[2-cyclohexyl-5-methylphenol], 4,4′-[(4-hydroxyphenyl)methylene]bis[2-cyclohexyl-5-methylphenol], 4,4′-[(3-methoxy-4-hydroxyphenyl)methylene]bis[2-cyclohexyl-5-methylphenol], 4,4′-[(3,4-dihydroxyphenyl)methylene]bis[2-methylphenol], 4,4′-[(3,4-dihydroxyphenyl)methylene]bis[2,6-dimethylphenol], and 4,4′-[(3,4-dihydroxyphenyl)methylene]bis[2,3,6-trimethylphenol]. These skeletons are used singly or in combination. A hydroxyl group-containing compound having the triarylmethane skeleton may be used as a quinone diazide compound obtained by ester-bonding a sulfonic acid of naphthoquinone diazide to the hydroxyl group-containing compound.

In the present invention, when the thermally coloring compound is contained, the content is preferably 5 to 80 parts by mass, and particularly preferably 10 to 60 parts by mass based on 100 parts by mass of the alkali-soluble resin (a). When the content of the thermally coloring compound is 5 parts by mass or more, the transmittance of the cured object in the ultraviolet and visible light region can be reduced. When the content is 80 parts by mass or less, the heat resistance and the strength of the cured object can be maintained, and the water absorption rate can be reduced.

### <Radically Polymerizable Compound>

The resin composition of the present invention may contain a radically polymerizable compound. In particular, when the resin composition contains the photo initiator (c2), it is essential to contain a radically polymerizable compound. The term “radically polymerizable compound” refers to a compound having a plurality of ethylenic unsaturated double bonds in the molecule. At the time of exposure, radical polymerization of the radically polymerizable compound proceeds by radicals generated from the photo initiator (c2). As a result, the part irradiated with light is insolubilized, and thus, a negative pattern can be obtained. Furthermore, by the fact that the radically polymerizable compound is included, photocuring of the part irradiated with light is promoted to further improve the sensitivity. In addition, the crosslinking density after heat curing is improved, so that the hardness of the cured object can be improved.

The radically polymerizable compound is preferably a compound in which radical polymerization is likely to proceed and a (meth)acrylic group is included. From the viewpoint of the improvement in sensitivity upon exposure to light and the improvement in the hardness of the cured object, a compound having two or more (meth)acrylic groups in the molecule is more preferable. The radically polymerizable compound preferably has a double bond equivalent of 80 to 400 g/mol from the viewpoint of improving the sensitivity at the time of exposure and improving the hardness of the cured object.

Examples of the radically polymerizable compound can include trimethylolpropane tri(meth)acrylate, ditrimethylolpropane tri(meth)acrylate, ditrimethylolpropane tetra(meth)acrylate, pentaerythritol tri(meth)acrylate, pentaerythritol tetra(meth)acrylate, dipentaerythritol penta(meth)acrylate, dipentaerythritol hexa(meth)acrylate, tripentaerythritol hepta(meth)acrylate, tripentaerythritol octa(meth)acrylate, 2,2-bis[4-(3-(meth)acryloxy-2-hydroxypropoxy)phenyl]propane, 1,3,5-tris((meth)acryloxyethyl)isocyanuric acid, 1,3-bis((meth)acryloxyethyl)isocyanuric acid, 9,9-bis[4-(2-(meth)acryloxyethoxy)phenyl]fluorene, 9,9-bis[4-(3-(meth)acryloxypropoxy)phenyl]fluorene, and 9,9-bis(4-(meth)acryloxyphenyl)fluorene, and acid modified products, ethylene oxide modified products, and propylene oxide modified products of the above-described compounds.

The content of the radically polymerizable compound is preferably 15 mass % or more and more preferably 30 mass % or more in 100 mass % of the total of the alkali-soluble resin (a) and the radically polymerizable compound from the viewpoint of further improving the sensitivity and decreasing the taper angle. The content of the radically polymerizable compound is preferably 65 mass % or less and more preferably 50 mass % or less in 100 mass % of the total of the alkali-soluble resin (a) and the radically polymerizable compound from the viewpoint of further improving the heat resistance of the cured object and decreasing the taper angle.

### <Thermal Crosslinking Agent>

The resin composition of the present invention may contain a thermal crosslinking agent. The term “thermal crosslinking agent” refers to a compound having at least two thermally reactive functional groups such as an alkoxymethyl group, a methylol group, an epoxy group, and an oxetanyl group in the molecule. By containing a thermal crosslinking agent, the thermal crosslinking agent and the alkali-soluble resin (a) or the thermal crosslinking agents are crosslinked and the heat resistance, chemical resistance, and bending resistance of the cured object after heat curing can be improved.

Preferred specific examples of the compound having at least two alkoxymethyl groups or methylol groups can include DML-PC, DML-PEP, DML-OC, DML-OEP, DML-34X, DML-PTBP, DML-PCHP, DML-OCHP, DML-PFP, DML-PSBP, DML-POP, DML-MBOC, DML-MBPC, DML-MTrisPC, DML-BisOC-Z, DML-BisOCHP-Z, DML-BPC, DML-BisOC-P, DMOM-PC, DMOM-PTBP, DMOM-MBPC, TriML-P, TriML-35XL, TML-HQ, TML-BP, TML-pp-BPF, TML-BPE, TML-BPA, TML-BPAF, TML-BPAP, TMOM-BP, TMOM-BPE, TMOM-BPA, TMOM-BPAF, TMOM-BPAP, HML-TPPHBA, HML-TPHAP, HMOM-TPPHBA and HMOM-TPHAP (product names, manufactured by Honshu Kagaku Industry Co., Ltd.), and “NIKALAC” (a registered trademark) MX-290, “NIKALAC” MX-280, “NIKALAC” MX-270, “NIKALAC” MX-279, “NIKALAC” MW-100LM and “NIKALAC” MX-750LM (product names, manufactured by Sanwa Chemical Co., Ltd.).

Preferable examples of the compound having at least two epoxy groups can include “EPOLIGHT” (registered trademark) 40E, “EPOLIGHT” 100E, “EPOLIGHT” 200E, “EPOLIGHT” 400E, “EPOLIGHT” 70P, “EPOLIGHT” 200P, “EPOLIGHT” 400P, “EPOLIGHT” 1500NP, “EPOLIGHT” 80MF, “EPOLIGHT” 4000, and “EPOLIGHT” 3002 (all manufactured by Kyoeisha Chemical Co., Ltd.), “DENACOL” (registered trademark) EX-212L, “DENACOL” EX-214L, “DENACOL” EX-216L, and “DENACOL” EX-850L (all manufactured by Nagase ChemteX Corporation), GAN and GOT (all manufactured by Nippon Kayaku Co., Ltd.), “EPIKOTE” (registered trademark) 828, “EPIKOTE” 1002, “EPIKOTE” 1750, “EPIKOTE” 1007, YX8100-BH30, E1256, E4250, and E4275 (all manufactured by Japan Epoxy Resin Co., Ltd.), “EPICLON” (registered trademark) EXA-9583 and HP4032 (all manufactured by DIC Corporation), VG3101 (manufactured by Mitsui Chemicals, Inc.), “TEPIC” (registered trademark) S, “TEPIC” G, and “TEPIC” P (all manufactured by Nissan Chemical Industries, Ltd.), “DENACOL” EX-321L (manufactured by Nagase ChemteX Corporation), NC6000 (manufactured by Nippon Kayaku Co., Ltd.), “Epotohto” (registered trademark) YH-434L (manufactured byTohto KaseiCo., Ltd.), EPPN502H and NC3000 (manufactured by Nippon Kayaku Co., Ltd.), and “EPICLON” (registered trademark) N695 and HP7200 (all manufactured by DIC Corporation).

Examples of the compound having at least two oxetanyl groups can include ETERNACOLL EHO, ETERNACOLL OXBP, ETERNACOLL OXTP, and ETERNACOLL OXMA (all manufactured by Ube Industries, Ltd.), and oxetanized phenol novolac.

Two or more of the thermal crosslinking agents may be included in combination.

When the thermal crosslinking agent is contained, the content is preferably 1 mass % or more and 30 mass % or less based on 100 mass % of the total amount of the resin composition excluding the solvent. When the content of the thermal crosslinking agent is 1 mass % or more, the chemical resistance and the bending resistance of the cured object can be further enhanced. When the content of the thermal crosslinking agent is 30 mass % or less, the amount of outgas from the cured object can be further reduced, the long-term reliability of an organic EL display device can be further enhanced, and the storage stability of the resin composition is also excellent.

### <Solvent>

The resin composition of the present invention may contain a solvent. By containing the solvent, the resin composition can be in a varnish state and the coating property can be improved.

Examples of the solvent may include polar aprotic solvents such as γ-butyrolactone, ethers such as ethylene glycol monomethyl ether, ethylene glycol monoethyl ether, ethylene glycol mono-n-propyl ether, ethylene glycol mono-n-butyl ether, diethylene glycol monomethyl ether, diethylene glycol monoethyl ether, diethylene glycol mono-n-propyl ether, diethylene glycol mono-n-butyl ether, triethylene glycol monomethyl ether, triethylene glycol monoethyl ether, propylene glycol monomethyl ether, propylene glycol monoethyl ether, propylene glycol mono-n-propyl ether, propylene glycol mono-n-butyl ether, dipropylene glycol monomethyl ether, dipropylene glycol monoethyl ether, dipropylene glycol mono-n-propyl ether, dipropylene glycol mono-n-butyl ether, tripropylene glycol monomethyl ether, tripropylene glycol monoethyl ether, tetrahydrofuran, and dioxane, ketones such as acetone, methyl ethyl ketone, diisobutyl ketone, cyclohexanone, 2-heptanone, 3-heptanone, and diacetone alcohol, esters such as ethylene glycol monomethyl ether acetate, ethylene glycol monoethyl ether acetate, diethylene glycol monomethyl ether acetate, diethylene glycol monoethyl ether acetate, propylene glycol monomethyl ether acetate, propylene glycol monoethyl ether acetate, and ethyl lactate, other esters such as ethyl 2-hydroxy-2-methylpropionate, methyl 3-methoxypropionate, ethyl 3-methoxypropionate, methyl 3-ethoxypropionate, ethyl 3-ethoxypropionate, ethyl ethoxyacetate, ethyl hydroxyacetate, methyl 2-hydroxy-3-methylbutanoate, 3-methoxybutyl acetate, 3-methyl-3-methoxybutyl acetate, 3-methyl-3-methoxybutyl propionate, ethyl acetate, n-propyl acetate, i-propyl acetate, n-butyl acetate, i-butyl acetate, n-pentyl formate, i-pentyl acetate, n-butyl propionate, ethyl butyrate, n-propyl butyrate, i-propyl butyrate, n-butyl butyrate, methyl pyruvate, ethyl pyruvate, n-propyl pyruvate, methyl acetoacetate, ethyl acetoacetate, and ethyl 2-oxobutanoate, aromatic hydrocarbons such as toluene and xylene, amides such as N-methylpyrrolidone, N,N-dimethylformamide, N,N-dimethylacetamide, 3-methoxy-N,N-dimethylpropionamide, 3-butoxy-N,N-dimethylpropionamide, N,N-dimethylpropanamide, and N,N-dimethylisobutylamide, and 3-methyl-2-oxazolidinone. Two or more of these solvents may be contained.

The content of the solvent is not particularly limited but is preferably 100 to 3000 parts by mass and more preferably 150 to 2000 parts by mass based on 100 parts by mass of the total amount of the resin composition excluding the solvent. In 100 mass % of the total amount of the solvent, the proportion of a solvent having a boiling point of 180° C. or higher is preferably 20 mass % or less, and more preferably 10 mass % or less. By setting the proportion of the solvent having a boiling point of 180° C. or higher to 20 mass % or less, it is possible to further reduce the amount of outgas after heat curing and further enhance the long-term reliability of an organic EL apparatus.

### <Adhesion Promoter>

The resin composition of the present invention may contain an adhesion promoter. Specific examples of the adhesion promoter can include: a silane coupling agent such as vinyltrimethoxysilane, vinyltriethoxysilane, epoxycyclohexylethyltrimethoxysilane, 3-glycidoxypropyltrimethoxysilane, 3-glycidoxypropyltriethoxysilane, p-styryltrimethoxysilane, 3-aminopropyltrimethoxysilane, 3-aminopropyltriethoxysilane and N-phenyl-3-aminopropyltrimethoxysilane; a titanium chelating agent; an aluminum chelating agent; and a compound produced by reacting an aromatic amine compound with a silicon compound containing an alkoxy group. Two or more of these adhesion promoters may be contained. By containing these adhesion promoters, it is possible to enhance the development adhesion, in development or the like, of a resin film with an underlying substrate such as a silicon wafer, indium tin oxide (ITO), SiO₂, or silicon nitride. In this case, it becomes also possible to improve the resistance to oxygen plasma that is used for washing purposes or a UV ozone treatment. The content of the adhesion promoter is preferably 0.01 to 10 mass % in 100 mass % of the total amount of the resin composition excluding the solvent.

### <Surfactant>

The resin composition of the present invention may contain a surfactant. When the resin composition contains a surfactant, the wettability to the substrate can be improved. Examples of the surfactant can include fluorine-based surfactants such as SH series, SD series, and ST series manufactured by Toray Dow Corning, BYK series manufactured by BYK JAPAN K.K., KP series manufactured by Shin-Etsu Chemical Co., Ltd., DISFOAM series manufactured by NOF CORPORATION, “MEGAFACE (registered trademark)” series manufactured by DIC Corporation, Fluorad series manufactured by Sumitomo 3M Limited, “SURFLON (registered trademark)” series and “AsahiGuard (registered trademark)” series manufactured by Asahi Glass Co., Ltd., and POLYFOX series manufactured by OMNOVA Solutions Inc., and acryl-based and/or methacryl-based surfactants such as POLYFLOW series manufactured by Kyoeisha Chemical Co., Ltd. and “DISPARLON (registered trademark)” series manufactured by Kusumoto Chemicals, Ltd.

When the surfactant is contained, the content is preferably 0.001 to 1 mass % in 100 mass % of the total amount of the resin composition excluding the solvent.

### <Inorganic Particles>

The resin composition of the present invention may contain inorganic particles. Preferable specific examples of the inorganic particles can include silicon oxide, titanium oxide, barium titanate, alumina, and talc. The primary particle diameter of the inorganic particles is preferably 100 nm or less and more preferably 60 nm or less.

The content of the inorganic particles is preferably 5 to 90 mass % in 100 mass % of the total amount of the resin composition excluding the solvent.

### <All Chlorine Atoms and all Bromine Atoms>

In the resin composition of the present invention, a total mass of all chlorine atoms and all bromine atoms contained in the resin composition is preferably 150 ppm or less, more preferably 100 ppm or less, is still more preferably 2 ppm or less which is the detection lower limit of combustion ion chromatography, based on a total mass of solid contents of the resin composition. The total mass of solid contents of the resin composition refers to a mass obtained by excluding the mass of the solvent from the total mass of the resin composition. The lower limit of the total mass of all chlorine atoms and all bromine atoms is 0 ppm, and the detection lower limit of combustion ion chromatography or less is regarded as 0 ppm.

By setting the total amount of all chlorine atoms and all bromine atoms contained in the resin composition to 150 ppm or less based on the solid content of the resin composition, deterioration of an electrode or a light-emitting layer in an organic EL display device having the cured object obtained by curing the resin composition can be suppressed, and long-term reliability can be improved.

### <Method for Producing Resin Composition>

In the following, a method for producing the resin composition of the present invention will be described. For example, the resin composition of the present invention can be obtained by dissolving the xanthene compound (b), the alkali-soluble resin (a), and if necessary, the photosensitive compound (c), the colorant (d), the thermally coloring compound, the radically polymerizable compound, the thermal crosslinking agent, the solvent, the adhesion promoter, the surfactant, the inorganic particles, and the like.

Examples of the dissolving method include stirring and heating. In the case of heating, the heating temperature is preferably set within a range without impairing the performance of the resin composition, and typically to room temperature to 80° C. The order of dissolving the components is not particularly limited, and examples of the method include a method in which the compounds are dissolved in the order of ascending solubility. Components that are likely to generate bubbles at the time of stirring and dissolution, such as surfactants and some adhesion promoters, can be added last after dissolving other components to prevent poor dissolution of other components due to generation of bubbles.

The obtained resin composition is preferably filtered with the use of a filtration filter to remove dust and particles. The pore size of the filter is, for example, 0.5 μm, 0.2 μm, 0.1 μm, 0.07 μm, 0.05 μm, or 0.02 μm, but is not limited thereto. Examples of the material of the filtration filter include polypropylene (PP), polyethylene (PE), nylon (NY), and polytetrafluoroethylene (PTFE). Among the materials, polyethylene and nylon are preferable.

### <Method for Producing Cured Object>

A method for producing a cured object of the present invention includes the steps of: forming, on a substrate, a resin film formed of the resin composition containing a photosensitive compound (c) among the resin compositions of the present invention; exposing the resin film; developing the exposed resin film; and subjecting the developed resin film to heat treatment.

The step of forming, on a substrate, a resin film formed of the resin composition containing a photosensitive compound (c) among the resin compositions of the present invention will be described. In the present invention, the resin film can be obtained by applying a resin composition containing a photosensitive compound (c) among the resin compositions of the present invention to obtain a coating film of the resin composition, and drying the coating film.

Examples of the method of applying the resin composition of the present invention include a spin coating method, a slit coating method, a dip coating method, a spray coating method, and a printing method. Among the methods, the slit coating method is preferable because a coating liquid can be applied in a small amount to be advantageous for cost reduction. The amount of the coating liquid to be used in the slit coating method is, for example, about ⅕ to 1/10 of that in the spin coating method. As the slit nozzle used for application, slit nozzles put on the market from a plurality of manufacturers can be selected. Examples of the slit nozzle include “Linear Coater” manufactured by Dainippon Screen Mfg. Co., Ltd., “Spinless” manufactured by TOKYO OHKA KOGYO CO., LTD., “TSCoater” manufactured by Toray Engineering Co., Ltd., “Table Coater” manufactured by CHUGAI RO CO., LTD., “CS Series” and “CL Series” manufactured by Tokyo Electron Ltd., “In-line type slit coater” manufactured by CERMATRONICS BOEKI Co., Ltd., and “Head Coater HC series” manufactured by Hirata Corporation. The application speed is generally in the range of 10 mm/sec to 400 mm/sec. The coating film is usually formed so that the thickness of the dried film is 0.1 to 10 μm, and preferably 0.3 to 5 μm although the film thickness depends on, for example, the solid content concentration and the viscosity of the resin composition.

Prior to coating, the substrate to be coated with the resin composition may be pretreated with the adhesion promoter described above in advance. Examples of the method of pretreatment include a method in which the surface of the substrate is treated with a solution prepared by dissolving 0.5 to 20 mass % of the adhesion promoter in a solvent such as isopropanol, ethanol, methanol, water, tetrahydrofuran, propylene glycol monomethyl ether acetate, propylene glycol monomethyl ether, ethyl lactate, or diethyl adipate. Examples of the method of treating the surface of the substrate include a spin coating method, a slit die coating method, a bar coating method, a dip coating method, a spray coating method, and a steam treatment method.

After the application, vacuum drying treatment is performed if necessary.

The vacuum drying rate depends on the volume of the vacuum chamber, the capacity of the vacuum pump, the diameter of the pipe between the chamber and the pump, and the like, but, for example, is preferably set to a condition that the pressure in the vacuum chamber is reduced to 40 Pa after a lapse of 60 seconds in the absence of the coated substrate. The general vacuum drying time is often about 30 seconds to 100 seconds, and the ultimate pressure in the vacuum chamber at the end of vacuum drying is usually 100 Pa or less in the presence of the coated substrate. By setting the ultimate pressure to 100 Pa or less, it is possible to bring the coating film into a dry state in which stickiness of the surface of the coating film is reduced, and as a result, it is possible to suppress surface contamination and generation of particles in the subsequent substrate conveyance.

After the application or vacuum drying, the coating film is generally heated and dried. This step is also referred to as prebaking. For drying, a hot plate, an oven, an infrared ray, or the like is used. In the case of using a hot plate, the coating film is held and heated directly on the plate, or on a jig such as a proxy pin installed on the plate. The heating time is preferably 1 minute to several hours. The heating temperature depends on the kind and the purpose of the coating film, but is preferably 80° C. or higher, and more preferably 90° C. or higher from the viewpoint of promoting solvent drying at the time of prebaking. From the viewpoint of reducing the progress of curing at the time of prebaking, the heating temperature is preferably 150° C. or lower, and more preferably 140° C. or lower.

Next, the step of exposing the resin film will be described.

In the resin film containing the photosensitive compound (c), a pattern can be formed. For example, a desired pattern can be formed by irradiating the resin film with actinic rays through a photomask having the desired pattern for exposure and conducting development.

In the step of exposing the resin film, the photomask used for exposure is preferably a half-tone photomask having a light-transmitting portion, a light-shielding portion, and a semi-translucent portion. The exposure with the use of the half-tone photomask makes it possible to form a pattern which has a step shape after development. When a positive resin film is used, in a pattern having a step shape, a portion formed from the light-shielding portion corresponds to a thick film portion, and a portion formed from a halftone exposed portion irradiated with active actinic rays through the semi-translucent portion corresponds to a thin film portion. When the transmittance of the light-transmitting portion in the half-tone photomask is taken as 100%, the transmittance of the semi-translucent portion is preferably 5% or more and more preferably 10% or more. When the transmittance of the semi-translucent portion is within the above range, a step difference between the thick film portion and the thin film portion can be clearly formed. The transmittance of the semi-translucent portion is preferably 30% or less, preferably 25% or less, still more preferably 20% or less, and most preferably 15% or less. When the transmittance of the semi-translucent portion is within the above range, the film thickness of the thin film portion can be increased, and the optical density of the entire film can be increased even when a black cured object having a low optical density in visible light per film thickness of 1 μm is formed.

Examples of the chemical rays used for exposure include ultraviolet rays, visible light, electron beams, and X-rays. In the present invention, i-line (365 nm), h-line (405 nm), and g-line (436 nm) of a mercury lamp are preferably used. When the resin film has positive photosensitivity, the exposed portion is dissolved in the developer. When the resin film has negative photosensitivity, the exposed portion is cured and insolubilized in the developer.

Next, the step of developing the exposed resin film will be described.

After the exposure, the exposed portion is removed in the case of the resin film having positive photosensitivity, and the unexposed portion is removed in the case of the resin film having negative photosensitivity with a developer to form a desired pattern. As the developer, an aqueous solution of an alkaline compound is preferable, and examples of the alkaline compound include tetramethylammonium hydroxide, diethanolamine, diethylaminoethanol, sodium hydroxide, potassium hydroxide, sodium carbonate, potassium carbonate, triethylamine, diethylamine, methylamine, dimethylamine, dimethylaminoethyl acetate, dimethylaminoethanol, dimethylaminoethyl methacrylate, cyclohexylamine, ethylenediamine, and hexamethylenediamine. To the alkaline aqueous solution, one or more components may be added, and examples of the components include polar solvents such as N-methyl-2-pyrrolidone, N,N-dimethylformamide, N,N-dimethylacetamide, dimethylsulfoxide, γ-butyrolactone, and dimethylacrylamide, alcohols such as methanol, ethanol, and isopropanol, esters such as ethyl lactate and propylene glycol monomethyl ether acetate, and ketones such as cyclopentanone, cyclohexanone, isobutyl ketone, and methyl isobutyl ketone. Examples of the method of development include a spray method, a paddle method, an immersion method, and an ultrasonic method.

Next, the pattern formed by development is preferably rinsed with distilled water. The pattern may be rinsed with distilled water to which a component is added, and examples of the component include alcohols such as ethanol and isopropyl alcohol, and esters such as ethyl lactate and propylene glycol monomethyl ether acetate.

Next, the step of subjecting the developed resin film to heat treatment will be described.

After the development, the developed resin film is subjected to heat treatment to obtain a cured object.

The heat treatment temperature is preferably 180° C. or higher, more preferably 200° C. or higher, still more preferably 230° C. or higher, and particularly preferably 250° C. or higher from the viewpoint of further reducing the amount of outgas generated from the cured object. From the viewpoint of improving the film toughness of the cured object, the temperature is preferably 500° C. or lower, and more preferably 450° C. or lower. In this temperature range, the temperature may be raised stepwise or may be continuously raised. The time for the heat treatment is preferably 30 minutes or longer from the viewpoint of further decreasing the amount of outgas. The time for the heat treatment is preferably 3 hours or shorter from the viewpoint of improving the film toughness of the cured object. For example, a method of performing heat treatment at 150° C. and 250° C. for 30 minutes each, a method of performing heat treatment while linearly increasing the temperature from room temperature to 300° C. over a period of 2 hours, or the like are mentioned.

### <Cured Object>

A first aspect of the cured object of the present invention is a cured object obtained by curing the resin composition of the present invention. By subjecting the resin composition of the present invention to heat treatment, it is possible to remove components exhibiting low heat resistance and thus to further improve the heat resistance and chemical resistance. Particularly, when the resin composition of the present invention contains a polyimide precursor, a polybenzoxazole precursor, a copolymer thereof, or a copolymer thereof with a polyimide, it is possible to further improve the heat resistance and chemical resistance since the imide ring and oxazole ring are formed by the heat treatment.

In the present invention, by using the xanthene compound (b), the component (d-2), the component (d-1) and/or the thermally coloring compound, and if necessary, the component (d-3) in combination, the light shielding property of visible light can be enhanced, and a black cured object can be obtained. The heat treatment temperature is preferably 180° C. or higher, more preferably 200° C. or higher, still more preferably 230° C. or higher, and particularly preferably 250° C. or higher from the viewpoint of further reducing the amount of outgas generated from the cured object. From the viewpoint of improving the film toughness of the cured object, the temperature is preferably 500° C. or lower, and more preferably 450° C. or lower. In this temperature range, the temperature may be raised stepwise or may be continuously raised. The time for the heat treatment is preferably 30 minutes or longer from the viewpoint of further decreasing the amount of outgas. The time for the heat treatment is preferably 3 hours or shorter from the viewpoint of improving the film toughness of the cured object. For example, there are a method in which the heat treatment is conducted at 150° C. for 30 minutes and at 250° C. for 30 minutes and a method in which the heat treatment is conducted while linearly raising the temperature from room temperature to 300° C. over 2 hours.

A second aspect of the cured object of the present invention is a cured object containing a xanthene compound (b′) represented by Formula (2) (hereinafter, may referred to as the cured object of the second aspect).

### [Chemical Formula 10]

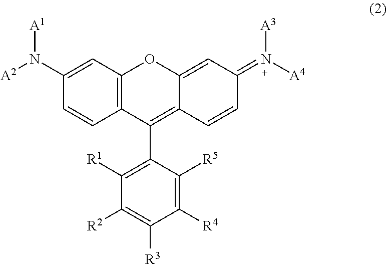

In Formula (2), A¹ to A⁴ each independently represent a hydrogen atom, an alkyl group having 1 to 10 carbon atoms, or an aryl group having 6 to 10 carbon atoms which may have an electron donating substituent. Provided that, at least three of A¹ to A⁴ are the aryl groups having 6 to 10 carbon atoms which may have an electron donating substituent, and at least one of the aryl groups having 6 to 10 carbon atoms which may have an electron donating substituent has an electron donating substituent. R¹ to R⁴ each independently represent a hydrogen atom, a halogen atom, a hydroxyl group, an alkoxy group, —SO₃H, —SO₃⁻, —SO₃NR⁶R⁷, —COOH, —COO⁻, —COOR⁸, —CONR⁹R¹⁰, or a monovalent hydrocarbon group having 1 to 20 carbon atoms. R⁵ represents a hydrogen atom, —SO₃H, —SO₃⁻, —SO₃NR⁶R⁷, —COOH, —COO⁻, —COOR⁸, or —CONR⁹R¹⁰. R⁶ to R¹⁰ each independently represent a monovalent hydrocarbon group having 1 to 20 carbon atoms. Provided that, the xanthene compound (b′) represented by Formula (2) is charge neutral or cationic.

When the cured object contains the xanthene compound (b′) represented by Formula (2), the light shielding property of the cured object in visible light can be enhanced. From the viewpoint of enhancing the light shielding property of the entire visible light, the cured object of the second aspect preferably further contains a colorant (d) other than Formula (2), and more preferably contains a colorant (d-2) having a maximum absorption wavelength in any of a range of 490 nm or more and less than 580 nm at 350 to 800 nm.

Other preferred embodiments of the xanthene compound (b′) represented by Formula (2) are the same as those of the xanthene compound (b) represented by Formula (1).

### Application Examples of Resin Composition and Cured Object

The resin composition containing the xanthene compound (b) and the cured object of the present invention are suitably used in a surface protective layer and an interlayer insulation layer of a semiconductor element, an insulation layer of an organic electroluminescence (hereinafter referred to as EL) element, a planarization layer of a thin film transistor (hereinafter referred to as TFT) substrate to be used for driving a display device in which an organic EL device is used, a wiring protective insulation layer of a circuit substrate, an on-chip microlens of a solid-state imaging element, and planarization layers for various display devices and solid-state imaging elements. For example, the resin composition and the cured object are suitable as a surface protective layer or an interlayer insulation layer in an MRAM having low heat resistance or in a promising next-generation memory such as a polymer memory (polymer ferroelectric RAM: PFRAM) or a phase change memory (phase change RAM: PCRAM or ovonics unified memory: OUM). The resin composition and the cured object can also be used in an insulation layer in a display device including a first electrode formed on a substrate and a second electrode provided so as to face to the first electrode, such as a liquid crystal display (LCD), an electrochemical display (ECD), an electroluminescent display (ELD), or a display device in which an organic electroluminescent element is used (organic electroluminescent apparatus). Hereinafter, an organic EL display device, a semiconductor device, and a semiconductor electronic component will be described as examples.

### <Organic EL Display Device>

The organic EL display device of the present invention includes a substrate, a drive circuit, a planarization layer, a first electrode, an insulation layer, a light-emitting layer, and a second electrode, in which the drive circuit, the planarization layer, the first electrode, the insulation layer, the light-emitting layer, and the second electrode are placed over the substrate, and the planarization layer and/or the insulation layer includes the cured object of the present invention. The substrate is a part of the organic EL display device.

In the organic EL display device of the present invention, the insulation layer includes the cured object of the present invention, and an optical density of the insulation layer in visible light per film thickness of 1 μm is preferably 0.5 to 1.5. When the OD value is 0.5 or more, the light shielding property can be improved by the cured object, so that in display devices such as organic EL display devices or liquid crystal display devices, it becomes possible to reduce visualization of electrode wirings or reduce external light reflection. Therefore, contrast in image display can be improved. When the OD value is 1.5 or less, the sensitivity upon exposure to light can be improved when a resin composition containing a photosensitive compound is formed.

When the insulation layer is a black film, the film thickness of the insulation layer is preferably 1.0 to 5.0 μm, more preferably 1.5 μm or more, and still more preferably 2.0 μm or more. By setting the black insulation layer within the above range, even in a black film having a low optical density in visible light per film thickness of 1 μm, the optical density of the entire film can be increased, and the effect of reducing external light reflection can be enhanced.

Taking an active matrix display device as an example, an active matrix display device includes a TFT and a wiring located on a side portion of the TFT and connected to the TFT that are provided on a substrate such as glass or a plastic, a planarization layer provided on the TFT and the wiring so as to cover the unevenness, and a display element provided on the planarization layer. The display element and the wiring are connected via a contact hole formed in the planarization layer. An organic EL display device is particularly preferable in which the substrate having a drive circuit includes a resin film because flexible organic EL display device are recently the mainstream. When the cured object obtained by curing the resin composition of the present invention is used as an insulation layer or a planarization layer in such a flexible display device, the cured object is particularly preferably used because bending resistance is excellent. The resin film is particularly preferably a polyimide from the viewpoint of improving the adhesion to a cured object obtained by curing the resin composition of the present invention.

The organic EL display device of the present invention preferably further includes a color filter having a black matrix in order to enhance the effect of reducing external light reflection. The black matrix preferably contains, for example, a resin such as an epoxy-based resin, an acrylic resin, a urethane-based resin, a polyester-based resin, a polyimide-based resin, a polyolefin-based resin, or a siloxane-based resin.

The black matrix contains a colorant. Examples of the colorant can include black organic pigments, mixed color organic pigments, and black inorganic pigments. Examples of the black organic pigments can include carbon black, perylene black aniline black, and benzofuranone-based pigments. Examples of the mixed color organic pigments can include pigments produced by mixing two or more pigments of a color of red, blue, green, purple, yellow, magenta, and/or cyan to make a pseudo black color. Examples of the black inorganic pigments can include graphite; fine particles of metals such as titanium, copper, iron, manganese, cobalt, chromium, nickel, zinc, calcium, and silver; metal oxides; metal composite oxides, metal sulfides, metal nitrides; metal oxynitrides; and metal carbides. Among them, carbon black, titanium nitride, and titanium carbide having high light shielding property, and composite particles of these and a metal such as silver are preferable.

The OD value of the black matrix is preferably 1.5 or more, more preferably 2.5 or more, and still more preferably 4.5 or more.

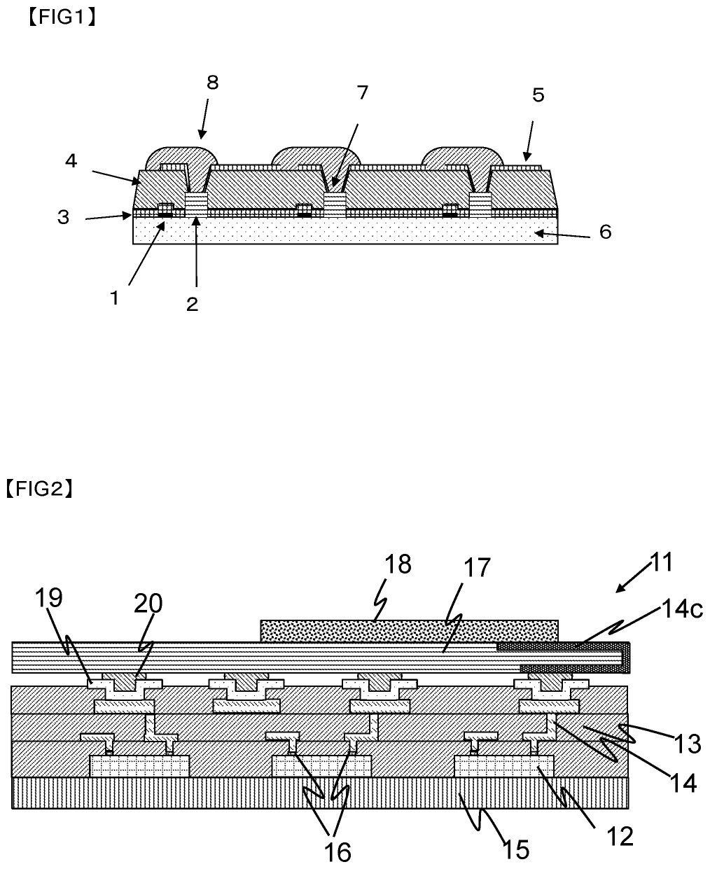

A cross-sectional view of an example of a TFT substrate is illustrated in FIG. 1. Bottom-gate type or top-gate type TFTs (thin film transistors) 1 are provided on a substrate 6 in rows and columns, and a TFT insulation layer 3 is formed so as to cover these TFTs 1. On the TFT insulation layer 3, a wiring 2 connected to the TFTs 1 is provided. On the TFT insulation layer 3, a planarization layer 4 is further provided in a state that the wiring 2 is embedded in the planarization layer 4. The planarization layer 4 is provided with contact holes 7 reaching the wiring 2. ITOs (transparent electrodes) 5 are formed on the planarization layer 4 in a state that ITOs 5 are connected to the wiring 2 via the contact holes 7, respectively. Here, each ITO 5 serves as an electrode of a display element (such as an organic EL element). Then, insulation layers 8 are formed so that the insulation layers 8 cover the peripheral edges of the ITOs 5, respectively. The organic EL element may be a top-emission organic EL device that emits light from the side opposite from the substrate 6, or may be a bottom-emission organic EL device that takes out light from the substrate 6 side. In this way, an active matrix organic EL display element is obtained in which each TFT 1 is connected to an organic EL device in order to drive the organic EL device.

The TFT insulation layer 3, the planarization layer 4 and/or the insulation layer 8 can be formed through a step of forming a resin film formed of the resin composition of the present invention as described above, a step of exposing the resin film, a step of developing the exposed resin film, and a step of subjecting the developed resin film to heat treatment. An organic EL display device can be obtained by a production method including these steps.

<Display Device Other than Organic EL Display Device>

A display device of the present invention includes at least a metal wiring, the cured object of the present invention, and a plurality of luminescent elements, in which each of the luminescent elements includes a pair of electrode terminals on either one surface, the pair of electrode terminals are connected to a plurality of the metal wirings extending in the cured object, and the plurality of the metal wirings are configured to retain electrical insulation properties by the cured object. The display device of the present invention refers to a display device other than the organic EL display device.

The display device will be described with FIG. 2 as an example of one aspect.

In FIG. 2, a display device 11 includes a plurality of luminescent elements 12 disposed on a counter substrate 15 and a cured object 13 disposed on the luminescent elements 12. The phrase “on the luminescent element” may be not only “on the surface of the luminescent element” but also “on the upper side of the support substrate or the luminescent element. In the aspect illustrated in FIG. 2, a configuration is exemplified in which a plurality of cured objects 13 are further laminated on the cured object 13 placed so as to be in contact with at least a part of the luminescent element 12 and a total of three layers are laminated, but the cured object 13 may be a single layer. The luminescent element 12 includes a pair of electrode terminals 16 on a surface opposite to a surface in contact with the counter substrate 15, and each electrode terminal 16 is connected to the metal wiring 14 extending in the cured object 13. When a plurality of metal wirings 14 extending in the cured object 13 are covered with the cured object 13, since the cured object 13 also functions as an insulation layer, the metal wirings are configured to retain electrical insulation properties. The fact that the metal wiring is configured to retain electrical insulation properties means that a portion requiring electrical insulation properties of the metal wiring is covered with a cured object obtained by curing the resin composition containing the alkali-soluble resin (a). In the present invention, the state that the insulation layer has electrical insulation properties means a state that the volume resistivity of the insulation layer is 10¹² Ω·cm or more. The luminescent element 12 is electrically connected to a drive element 18 added to a luminescent element driving substrate 17 provided at a position facing the counter substrate 15 through the metal wirings 14 and 14c, and light emission of the luminescent element 12 can be controlled. The luminescent element driving substrate 17 is electrically connected to the metal wiring 14 via, for example, a solder bump 20. In order to prevent diffusion of metal such as the metal wiring 14, a barrier metal 19 may be disposed.

It is preferable that the cured object 13 is preferably black and has an OD value of 0.5 to 1.5 in visible light per film thickness of 1 μm of the insulation layer. When the OD value is 0.5 or more, the light shielding property can be improved by the cured object, so that in display devices such as organic EL display devices or liquid crystal display devices, it becomes possible to reduce visualization of electrode wirings or reduce external light reflection. Therefore, contrast in image display can be improved. When the OD value is 1.5 or less, the sensitivity upon exposure to light can be improved when a resin composition containing a photosensitive compound is formed.

### EXAMPLES

Hereinbelow, the present invention will be described by way of examples and others. However, the present invention is not limited by the examples. Each evaluation in Examples was performed with the following method.

### (1) Evaluation of Maximum Absorption Wavelength at 350 to 800 nm of Xanthene Compound (b)

A varnish A of the resin composition containing the xanthene compound (b) and a varnish B of the resin composition not containing the xanthene compound (b) obtained in each of Examples and Comparative Examples were applied onto a glass substrate of 5 cm square by spin coating so that the film thickness after the heat treatment (curing) was 1.5 μm, and prebaked at 120° C. for 120 seconds to obtain corresponding prebaked films A and B. Transmission spectra of the prebaked film A and the prebaked film B thus obtained at a wavelength of 300 nm to 800 nm were measured using an ultraviolet-visible spectrophotometer MultiSpec-1500 (manufactured by SHIMADZU CORPORATION). Next, the transmission spectrum of the prebaked film B was converted into absorbance and then subtracted from the transmission spectrum of the prebaked film A to obtain a transmission spectrum derived from the xanthene compound (b), and the maximum absorption wavelength at 350 to 800 nm was determined as “A” if 600 nm or more, “B” if 580 nm or more and less than 600 nm, and “C” if less than 580 nm.

### (2) Evaluation of Heat Resistance of Xanthene Compound (b)

The prebaked film A and the prebaked film B obtained in the same manner as in (1) were each cut into two pieces, the first piece was not treated at all, and the second piece was subjected to heat treatment at 230° C. for 1 hour in the air atmosphere using an inert oven CLH-21CD-S (manufactured by Koyo Thermo Systems Co., Ltd.) to prepare corresponding cured objects A and B. Thereafter, the transmission spectra of the prebaked film and the cured object at a wavelength of 300 nm to 800 nm were measured in the same manner as in (1), and the transmission spectra of the corresponding prebaked film B and cured film B were converted into absorbance and then subtracted from the transmission spectra of the prebaked film A and cured film A to obtain transmission spectra of the prebaked film and cured film derived from the xanthene compound (b). The absorbance at the maximum absorption wavelength was calculated from the transmission spectra of the obtained prebaked film and cured film derived from the xanthene compound (b), and the absorbance change rate (absorbance of the cured object derived from the xanthene compound (b)/absorbance of the prebaked film derived from the xanthene compound (b)) (%) was calculated. The absorbance change rate was determined as “A” if 90% or more, “B” if less than 90% and 75% or more, and “C” if less than 75%.

### (3) Evaluation of Sensitivity of Resin Composition

The varnish obtained in each of Examples and Comparative Examples was applied onto an 8-inch silicon wafer with a spin coating method using a coating/development apparatus ACT-8 (manufactured by Tokyo Electron Ltd.), and the resulting product was baked at 120° C. for 2 minutes to prepare a prebaked film having a film thickness of 4.0 μm. The film thickness was measured using a stylus profiler (P-15; manufactured by KLA Corporation). Then, the prebaked film was exposed at exposure energy increased by 5 mJ/cm² in the range of 50 to 300 mJ/cm² through a mask having a pattern of a 10 μm hole using an exposure machine i-line stepper NSR-2005i9C (manufactured by NIKON CORPORATION). After the exposure, the exposed prebaked film was developed with the development apparatus of ACT-8 using a 2.38 mass % tetramethylammonium aqueous solution (hereinafter referred to as TMAH, manufactured by TAMA CHEMICALS CO., LTD.) as a developer until the amount of film loss of the unexposed portion reached 0.5 μm, then rinsed with distilled water, and shaken off and dried to obtain a pattern.

The obtained pattern was observed using an FPD microscope MX 61 (manufactured by OLYMPUS CORPORATION) at a magnification of 20, and the aperture diameter of the hole was measured. The minimum exposure energy at which the aperture diameter of the contact hole reached 10 μm was determined and regarded as the sensitivity. The sensitivity was determined as “A” if less than 120 mJ/cm², “B” if 120 mJ/cm² or more and less than 150 mJ/cm², and “C” if 150 mJ/m² or more.

### (4) Evaluation of OD Value Per 1 μm of Resin Composition

The varnish obtained in each of Examples and Comparative Examples was applied onto a glass substrate of 5 cm square by spin coating so that the film thickness after the heat treatment (curing) was 2.0 μm, and prebaked at 120° C. for 120 seconds to prepare a prebaked film. Thereafter, the prebaked film was cured at 230° C. for 60 minutes in the air atmosphere using a high-temperature clean oven INH-9CD-S manufactured by Koyo Thermo Systems Co., Ltd. to prepare a cured film. The film thickness of the cured film was measured using a stylus profiler (P-15; manufactured by KLA Corporation). The OD value of the cured film thus obtained was measured using an optical densitometer (361T; manufactured by X-Rite, Inc.). The obtained OD value was divided by the film thickness of the cured film to obtain an OD value per 1 μm (OD value per 1 μm=OD value/the film thickness of the cured film). The OD value per 1 μm was determined as “A” if 0.70 or more, “B” if less than 0.70 and 0.50 or more, and “C” if less than 0.50.

### (5) Evaluation of Change Amount of OD Value by Repeated Curing of Resin Composition

The cured film obtained in (4) was cured at 230° C. for 60 minutes in the air atmosphere using the high-temperature clean oven INH-9CD-S manufactured by Koyo Thermo Systems Co., Ltd. again to prepare a cured film subjected to curing twice. The film thickness and the OD value of the cured film were measured in the same manner as in (4), and the obtained OD value was divided by the film thickness of the cured film to calculate an OD value per 1 μm after repeated curing. The change amount of the OD value by the repeated curing was determined as “A” if less than 0.05, “B” if less than 0.10 and 0.05 or more, and “C” if 0.10 or more. However, even when the change amount of the OD value was less than 0.10, a case where the OD value of (4) was less than 0.50 was determined as “C”.

### (6) Evaluation of Frozen Storage Stability of Resin Composition

Using a coating and developing apparatus “CLEAN TRACK ACT-12” manufactured by Tokyo Electron Co., Ltd., each varnish filtered and then stored still in a freezer at −18° C. for 60 days was applied onto a 12-inch Si wafer and dried on a hot plate at 100° C. for 3 minutes to obtain a photosensitive resin film having a film thickness of 1000 nm. As for the obtained photosensitive resin film, the number of foreign matters having a size of 0.27 μm or more was measured with a wafer surface inspection apparatus “WM-10” manufactured by TOPCON CORPORATION. The measurement area was an area of about 201 cm² inside a circle having a radius of 8 cm from the center of the wafer, and the number of foreign matters (defect density) per 1 cm² of the coating film was determined. The defect density per one substrate was determined as “A” if less than 1.00/cm², “B” if 1.00/cm² or more and less than 3.00/cm², and “C” if 3.00/cm² or more.

### (7) Analysis of Xanthene Compound (b) in Cured Film by TOF-SIMS

For the obtained cured film, the film surface was cleaned with etching ions, and then TOF-SIMS analysis was performed. The TOF-SIMS apparatus used for analysis and measurement conditions are as follows.
Apparatus: “TOF.SIMS5” manufactured by ION-TOF Company
Primary ion: Bi₃_{⁺⁺}
Primary ion acceleration voltage: 30 kV
Primary ion current: 0.1 pA
Etching ion: Ar gas cluster ion
Etching ion acceleration voltage: 5.0 kV
Measurement range: 200 μm×200 μm.

### Synthesis Example 1 Synthesis of Hydroxyl Group-Containing Diamine Compound (α)

2,2-bis(3-amino-4-hydroxypheny)hexafluoropropane (hereinafter, referred to as BAHF) (18.3 g, 0.05 mol) was dissolved in 100 mL of acetone and 17.4 g of propylene oxide (0.3 mol) and the mixture was cooled to −15° C. A solution prepared by dissolving 3-nitrobenzoyl chloride (20.4 g (0.11 mol)) in acetone (100 mL) was added dropwise to the cooled solution. After completion of dropping, the mixture was reacted at −15° C. for 4 hours, and then the temperature was returned to room temperature. The precipitated white solid was separated by filtration and vacuum-dried at 50° C.

The solid material (30 g) was placed in a 300-mL stainless autoclave and then dispersed in methyl cellosolve (250 mL), and 5 mass % palladium-carbon (2 g) was added to the solution. Hydrogen was introduced thereinto with a balloon, and a reduction reaction was performed at room temperature. After about 2 hours, it was confirmed that the balloon did not deflate anymore, and the reaction was terminated. After the termination of the reaction, a palladium compound as a catalyst was removed by filtration, and the mixture was concentrated using a rotary evaporator, thereby obtaining a hydroxyl group-containing diamine compound (α) represented by the following formula.

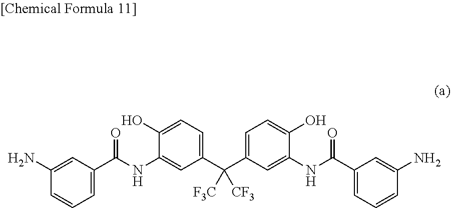 [SMILES: Nc1cccc(C(=O)Nc2cc(C(c3ccc(O)c(NC(=O)c4cccc(N)c4)c3)(C(F)(F)F)C(F)(F)F)ccc2O)c1]

### Synthesis Example 2 Synthesis of Quinone Diazide Compound (c-1)

Under a dried nitrogen stream, TrisP-PA (a product name, manufactured by Honshu Kagaku Industry Co., Ltd.) (21.22 g (0.05 mol)) and 5-naphthoquinone diazide sulfonic acid chloride (26.87 g (0.10 mol)) were dissolved in 1,4-dioxane (450 g) and the temperature of the resultant solution was restored to room temperature. To this solution was added dropwise a mixture of 1,4-dioxane (50 g) and triethylamine (15.18 g) while avoiding the increase in temperature of the inside of the system to 35° C. or more. After the dropwise addition, the resultant mixture was stirred at 30° C. for 2 hours. A triethylamine salt was filtered out from the solution, and a filtrate was introduced into water. Then, the precipitated precipitate was collected by filtration. The precipitate was dried with a vacuum dryer to obtain a quinone diazide compound (c-1) represented by the following formula.

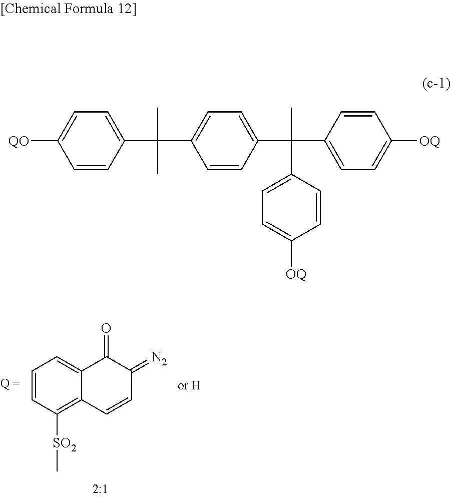

### Synthesis Example 3 Synthesis of Alkali-Soluble Resin (a-1)

Under a stream of dry nitrogen, 31.0 g (0.10 mol) of 3,3′,4,4′-diphenyl ether tetracarboxylic dianhydride (hereinafter, referred to as ODPA) was dissolved in 500 g of 1-methyl-2-pyrrolidone (hereinafter, referred to as NMAP). The hydroxyl group-containing diamine compound (α) (45.35 g (0.075 mol)) produced in Synthesis Example 1 and 1,3-bis(3-aminopropyl)tetramethyldisiloxane (hereinafter, referred to as SiDA) (1.24 g (0.005 mol)) were added together with NMP (50 g) to the solution, and the resultant mixture was reacted at 40° C. for 2 hours. Subsequently, 3-aminophenol (hereinafter, referred to as MAP) (4.36 g (0.04 mol)) that served as an end-capping agent was added together with NMP (5 g) to the reaction solution, and the resultant mixture was reacted at 50° C. for 2 hours. Then, a solution prepared by diluting 32.39 g (0.22 mol) of N,N-dimethylformamide diethyl acetal with 50 g of NMP was added. After the addition, the solution was stirred at 50° C. for 3 hours. After the completion of the stirring, the solution was cooled to room temperature and the solution was introduced into water (3 L) to produce white precipitates. This precipitate was collected by filtration, washed with water three times, and then dried in a vacuum dryer at 80° C. for 24 hours, thereby obtaining polyimide precursor (a-1) which was an alkali-soluble resin.

### Synthesis Example 4 Synthesis of Xanthene Compound (b-1)

A mixture of 20.26 g (0.05 mol) of the compound represented by (β) in the following Reaction Formula [1], 120 g of ethylene glycol, and 20.58 g (0.15 mol) of 4-ethoxyaniline was heated and stirred at 120° C. for 18 hours. After completion of the reaction, the reaction solution was allowed to cool to room temperature, and the reaction solution was added dropwise to 450 g of 17.5 mass % hydrochloric acid at 0 to 10° C., followed by stirring for 1 hour. Thereafter, the precipitate was collected by filtration, washed with pure water at 80° C., and dried at 60° C. for 24 hours to obtain a xanthene compound (b-1-1) in which two nitrogen atoms were substituted with an aryl group.

Next, a mixture of 24.27 g (0.04 mol) of the obtained compound (b-1-1), 150 g of 1-methyl-2-pyrrolidone, 1.3 g of copper powder, 8.3 g of potassium carbonate, and 19.84 g (0.08 mol) of 4-iodophenitol was heated and stirred at 150° C. for 12 hours. After completion of the reaction, the reaction solution was filtered to remove insoluble matters, and the reaction solution was added dropwise to 540 g of 17.5 mass % hydrochloric acid at 0 to 10° C., followed by stirring for 1 hour. Thereafter, the precipitate was collected by filtration and dried at 60° C. for 24 hours to obtain a xanthene compound (b-1) in which three nitrogen atoms were substituted with an aryl group. The obtained compound was subjected to LC-MS analysis using LC-MS2020 (manufactured by SHIMADZU CORPORATION) and confirmed to be a target compound.

LC-MS (ESI, posi): m/z 727 [M+H]⁺

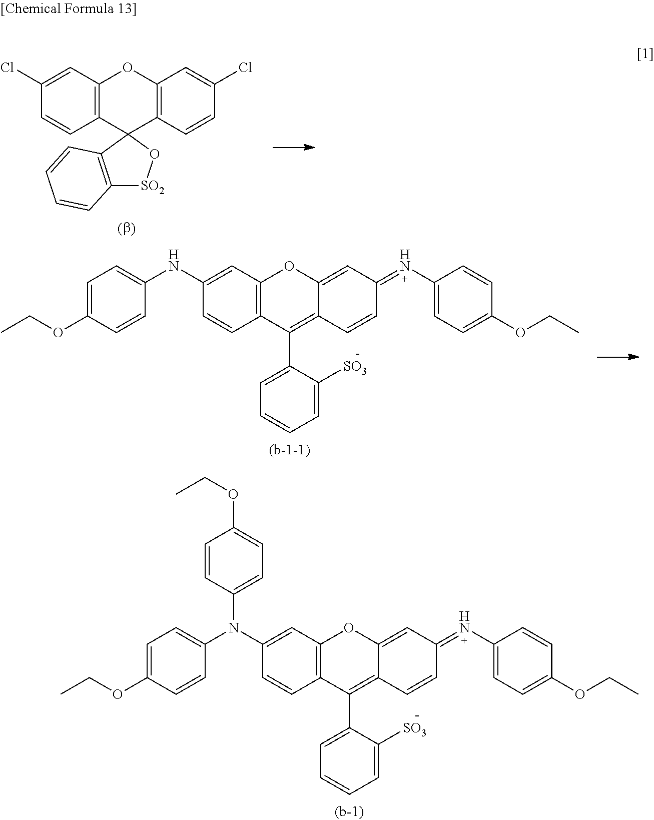

### Synthesis Example 5 Synthesis of Xanthene Compound (b-2)

A mixture of 18.46 g (0.05 mol) of the compound represented by (γ) in the following Reaction Formula [2], 120 g of sulfolane, 13.63 g of zinc chloride, and 20.58 g (0.15 mol) of 4-ethoxyaniline was heated and stirred at 170° C. for 8 hours. After completion of the reaction, the reaction solution was allowed to cool to room temperature, and the reaction solution was added dropwise to 450 g of 17.5 mass % hydrochloric acid at 0 to 10° C., followed by stirring for 1 hour. Subsequently, the precipitate was collected by filtration and added to 500 g of a 5 mass % sodium carbonate aqueous solution, and the mixture was stirred for 1 hour, collected by filtration, washed with pure water, and dried at 60° C. for 24 hours to obtain a xanthene compound (b-2-1) in which two nitrogen atoms were substituted with an aryl group.

Next, a mixture of 22.83 g (0.04 mol) of the obtained compound (b-2-1), 150 g of 1-methyl-2-pyrrolidone, 1.3 g of copper powder, 8.3 g of potassium carbonate, and 19.84 g (0.08 mol) of 4-iodophenitol was heated and stirred at 150° C. for 12 hours. After completion of the reaction, the reaction solution was filtered to remove insoluble matters, and the reaction solution was added dropwise to 450 g of 17.5 mass % hydrochloric acid at 0 to 10° C., followed by stirring for 1 hour. Thereafter, the precipitate was collected by filtration and dried at 60° C. for 24 hours to obtain a xanthene compound (b-2) in which four nitrogen atoms were substituted with an aryl group. The obtained compound was subjected to LC-MS analysis using LC-MS2020 (manufactured by SHIMADZU CORPORATION) and confirmed to be a target compound.

LC-MS (ESI, posi): m/z 811 [M+H]⁺

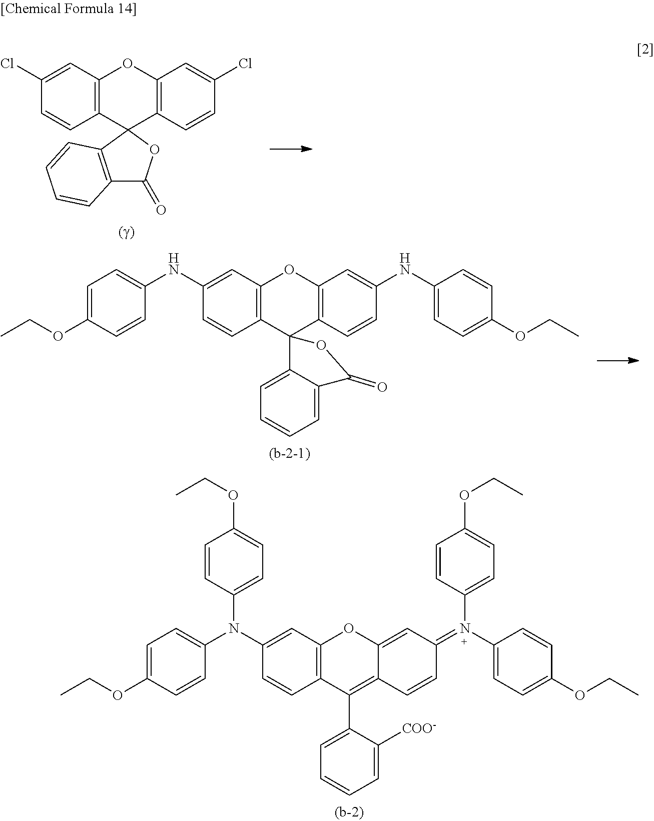

### Synthesis Example 6 Synthesis of Xanthene Compound (b-3)

In the following Reaction Formula [3], 1.69 g (0.011 mol) of phosphorus oxychloride was added dropwise to a mixture of 8.10 g (0.01 mol) of the xanthene compound (b-2) obtained in Synthesis Example 5, 2.54 g (0.015 mol) of diphenylamine, 10.11 g (0.1 mol) of triethylamine, and 150 g of 1,2-dichloroethane at room temperature, and heated and stirred at 85° C. for 3 hours. After completion of the reaction, the reaction solution was allowed to cool to room temperature, and then the reaction solution was placed in 300 g of pure water, and extracted with 100 g of chloroform. The organic layer was washed with 150 g of 4 mol/L hydrochloric acid and 150 g of pure water, and then the solvent was distilled off to obtain a xanthene compound (b-3) in which the xanthene compound (b-2) was amidated. The obtained compound was subjected to LC-MS analysis using LC-MS2020 (manufactured by SHIMADZU CORPORATION) and confirmed to be a target compound.

LC-MS (ESI, posi): m/z 963 [M+H]⁺

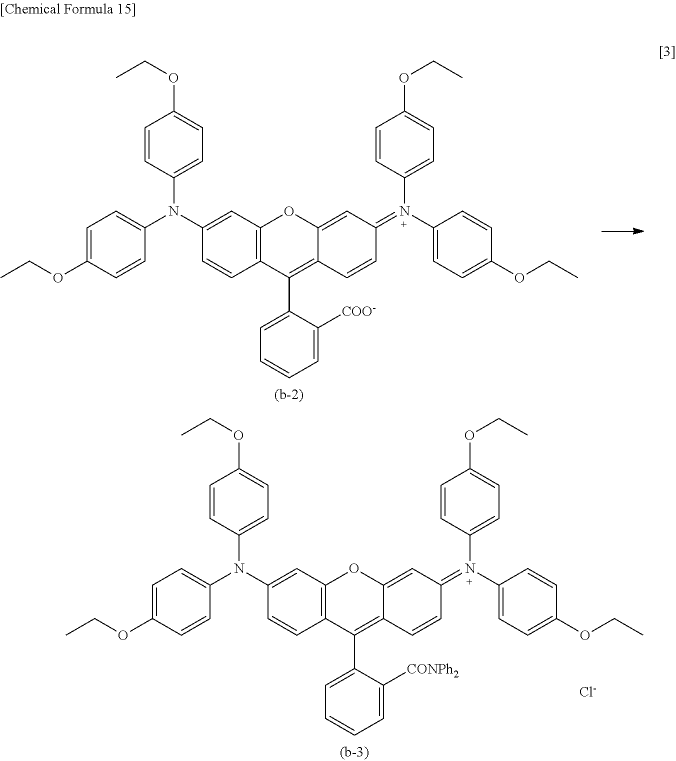

### Synthesis Example 7 Synthesis of Xanthene Compound (b-4)

In the following Reaction Formula [5], a mixture of 22.83 g (0.04 mol) of the compound (b-2-1) obtained in the same manner as in Synthesis Example 5, 150 g of 1-methyl-2-pyrrolidone, 1.3 g of copper powder, 8.3 g of potassium carbonate, and 17.43 g (0.08 mol) of 3-iodotoluene was heated and stirred at 150° C. for 12 hours. After completion of the reaction, the reaction solution was filtered to remove insoluble matters, and the reaction solution was added dropwise to 450 g of 17.5 mass % hydrochloric acid at 0 to 10° C., followed by stirring for 1 hour. Thereafter, the precipitate was collected by filtration and dried at 60° C. for 24 hours to obtain a xanthene compound in which four nitrogen atoms were substituted with an aryl group. To a mixture of 7.51 g (0.01 mol) of the obtained xanthene compound, 2.54 g (0.015 mol) of diphenylamine, 10.11 g (0.1 mol) of triethylamine, and 150 g of 1,2-dichloroethane, 1.69 g (0.011 mol) of phosphorus oxychloride was added dropwise at room temperature, and heated and stirred at 85° C. for 3 hours. After completion of the reaction, the reaction solution was allowed to cool to room temperature, and then the reaction solution was placed in 300 g of pure water, and extracted with 100 g of chloroform. The organic layer was washed with 150 g of 4 mol/L hydrochloric acid and 150 g of pure water, and then the solvent was distilled off to obtain an amidated xanthene compound (b-4). The obtained compound was subjected to LC-MS analysis using LC-MS2020 (manufactured by SHIMADZU CORPORATION) and confirmed to be a target compound.

LC-MS (ESI, posi): m/z 903 [M+H]⁺

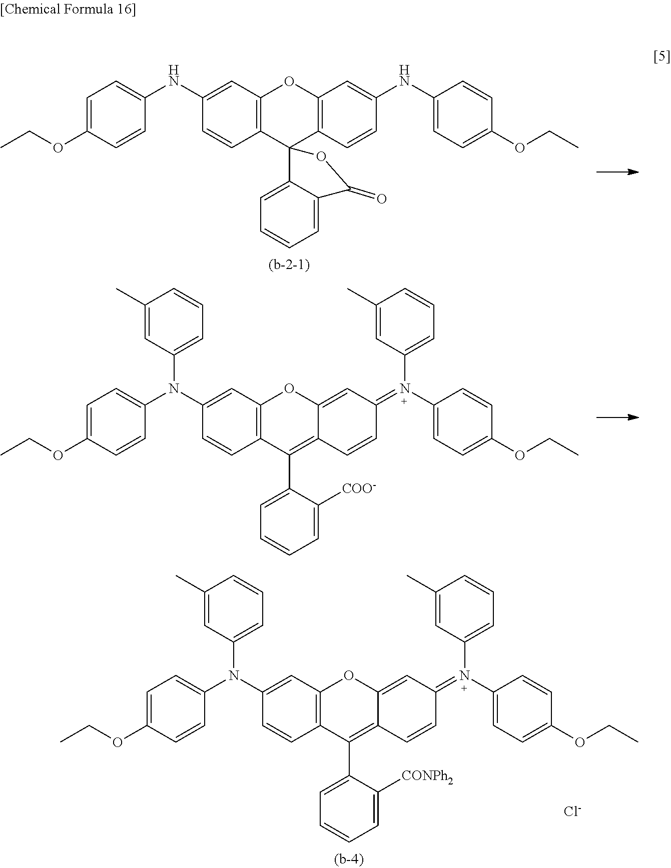

### Synthesis Example 8 Synthesis of Xanthene Compound (b-5)

In the following Reaction Formula [6], 9.39 g (0.01 mol) of the compound (b-4) obtained in the same manner as in Synthesis Example 7 was dissolved in 150 g of N,N-dimethylformamide (DMF), 2.91 g (0.015 mol) of sodium p-toluenesulfonate was added, and the mixture was heated and stirred at 40° C. for 3 hours. After the reaction solution was allowed to cool to room temperature, the reaction solution was poured into 1000 g of pure water, and precipitated crystals were collected by filtration, washed with water, and then dried at 60° C. for 24 hours to obtain a xanthene compound (b-5) in which counter ions of (b-4) were exchanged. The obtained compound was subjected to LC-MS analysis using LC-MS2020 (manufactured by SHIMADZU CORPORATION) and confirmed to be a target compound.

LC-MS (ESI, posi): m/z 903 [M+H]⁺

LC-MS (ESI, nega): m/z 171 [M]⁻

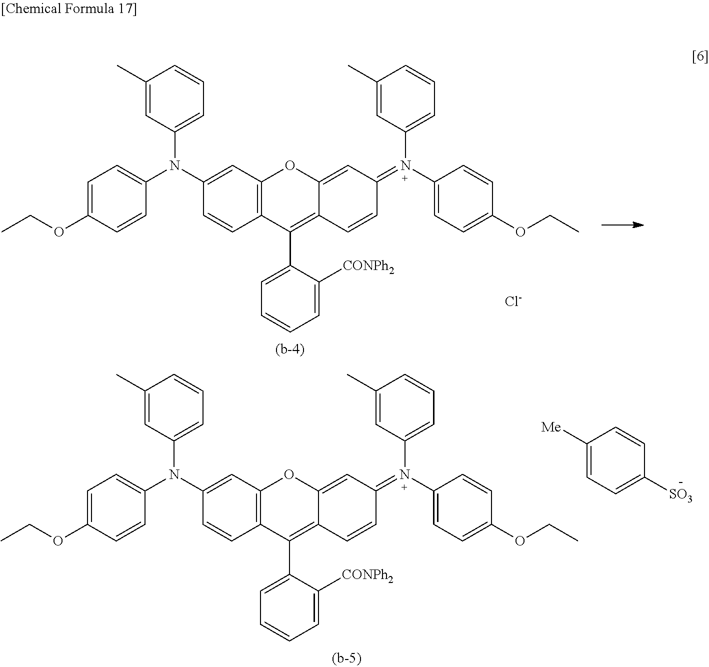

### Synthesis Example 9 Synthesis of Xanthene Compound (b-6)

A xanthene compound (b-6) in which counter ions of (b-4) were exchanged was obtained in the same manner as in Synthesis Example 8 except that 2.91 g (0.015 mol) of sodium p-toluenesulfonate was changed to 5.23 g (0.015 mol) of sodium laurylbenzenesulfonate in the following Reaction Formula [7]. The obtained compound was subjected to LC-MS analysis using LC-MS2020 (manufactured by SHIMADZU CORPORATION) and confirmed to be a target compound.

LC-MS (ESI, posi): m/z 903 [M+H]⁺

LC-MS (ESI, nega): m/z 325 [M]⁻

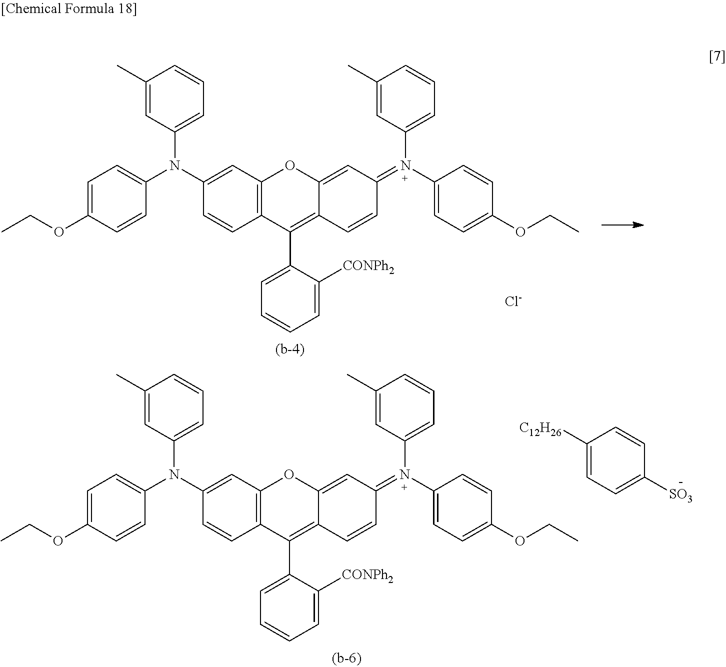

### Synthesis Example 10 Synthesis of Xanthene Compound (b-7)

An amidated xanthene compound (b-7) was obtained in the same manner as in Synthesis Example 7 except that 2.54 g (0.015 mol) of diphenylamine was changed to 1.28 g (0.015 mol) of piperidine in the following Reaction Formula [8].

The obtained compound was subjected to LC-MS analysis using LC-MS2020 (manufactured by SHIMADZU CORPORATION) and confirmed to be a target compound.

LC-MS (ESI, posi): m/z 819 [M+H]⁺

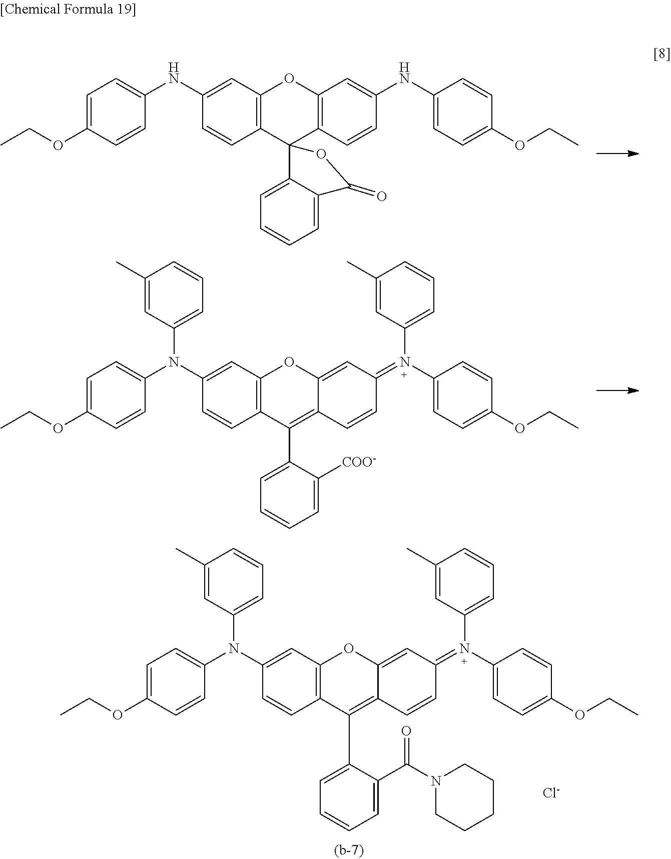

### Synthesis Example 11 Synthesis of Xanthene Compound (b-8)

A xanthene compound (b-8) in which a counter ion of (b-4) was exchanged was obtained in the same manner as in Synthesis Example 5 except that 2.91 g (0.015 mol) of sodium p-toluenesulfonate was changed to 2.58 g (0.015 mol) of sodium trifluoromethanesulfonate in the following Reaction Formula [10].

The obtained compound was subjected to LC-MS analysis using LC-MS2020 (manufactured by SHIMADZU CORPORATION) and confirmed to be a target compound.

LC-MS (ESI, posi): m/z 903 [M+H]⁺

LC-MS (ESI, nega): m/z 149 [M]⁻

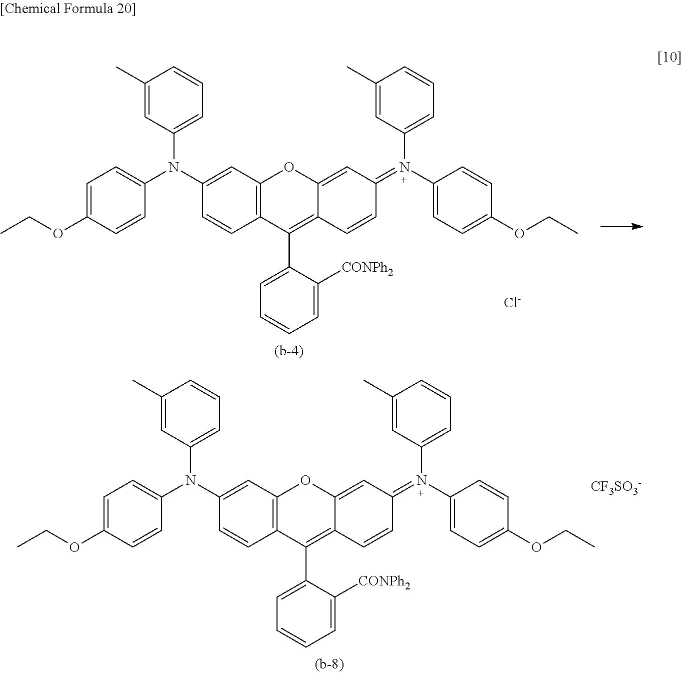

### Synthesis Example 12 Synthesis of Xanthene Compound (b-9)

A xanthene compound (b-9) in which counter ions of (b-4) were exchanged was obtained in the same manner as in Synthesis Example 8 except that 2.91 g (0.015 mol) of sodium p-toluenesulfonate was changed to 5.13 g (0.015 mol) of sodium tetraphenylborate in the following Reaction Formula [11].

The obtained compound was subjected to LC-MS analysis using LC-MS2020 (manufactured by SHIMADZU CORPORATION) and confirmed to be a target compound.

LC-MS (ESI, posi): m/z 903 [M+H]⁺

LC-MS (ESI, nega): m/z 319 [M]⁻

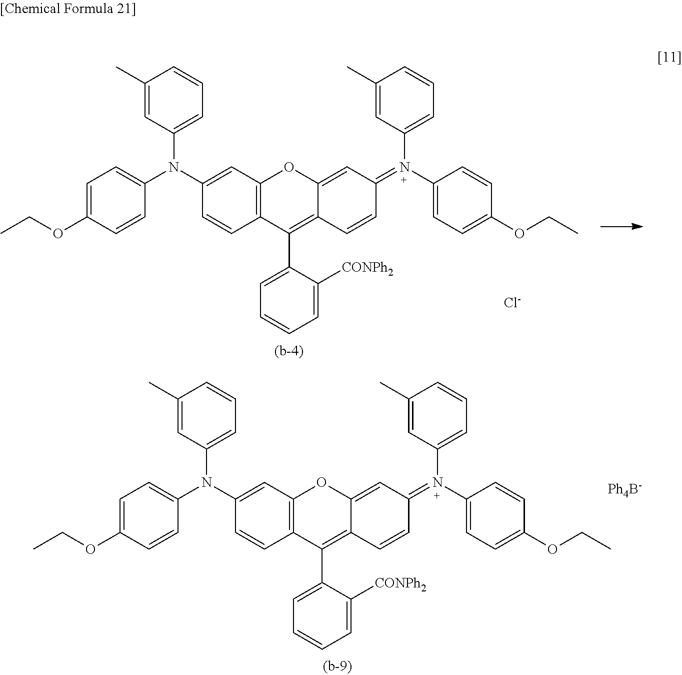

### Synthesis Example 13 Synthesis of Xanthene Compound (b-10)

A xanthene compound (b-10) in which a counter ion of (b-4) was exchanged was obtained in the same manner as in Synthesis Example 8 except that 2.91 g (0.015 mol) of sodium p-toluenesulfonate was changed to 4.78 g (0.015 mol) of potassium bis(trifluoromethanesulfonyl)imide in the following Reaction Formula [12].

The obtained compound was subjected to LC-MS analysis using LC-MS2020 (manufactured by SHIMADZU CORPORATION) and confirmed to be a target compound.

LC-MS (ESI, posi): m/z 903 [M+H]⁺

LC-MS (ESI, nega): m/z 280 [M]⁻

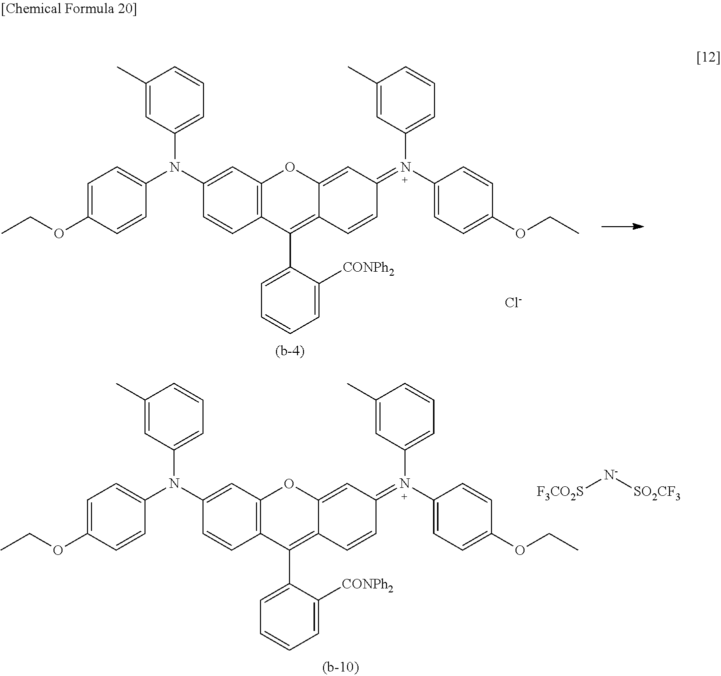

### Synthesis Example 11 Synthesis of Xanthene Compound (b-11)

A xanthene compound (b-11-1) in which four nitrogen atoms were substituted with an aryl group was obtained in the same manner as in Synthesis Example 5 except that 20.58 g (0.15 mol) of 4-ethoxyaniline was changed to 16.07 g (0.15 mol) of p-toluidine in the following Reaction Formula [13]. Next, a xanthene compound (b-11) in which the xanthene compound (b-11-1) was amidated was obtained in the same manner as in Synthesis Example 6 except that 8.10 g (0.01 mol) of the xanthene compound (b-2) was changed to 6.90 g (0.01 mol) of the obtained xanthene compound (b-11-1).

The obtained compound was subjected to LC-MS analysis using LC-MS2020 (manufactured by SHIMADZU CORPORATION) and confirmed to be a target compound.

LC-MS (ESI, posi): m/z 842 [M+H]⁺

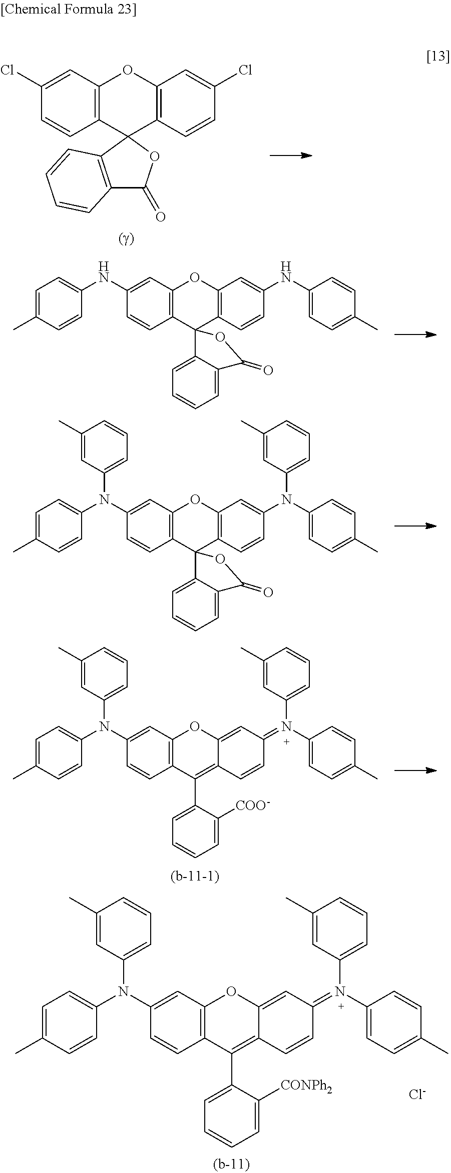

### Comparative Synthesis Example 1 Synthesis of Xanthene Compound (1)

A mixture of 20.26 g (0.05 mol) of the compound represented by (β) in the following Reaction Formula [4], 120 g of 2-propanol, and 7.3 g (0.06 mol) of 2,6-dimethylaniline was heated and stirred at 80° C. for 15 hours. After completion of the reaction, the reaction solution was allowed to cool to room temperature, and the reaction solution was added dropwise to 450 g of 17.5 mass % hydrochloric acid, followed by stirring at room temperature for 1 hour. Thereafter, the precipitate was collected by filtration, washed with pure water at 80° C., and dried at 60° C. for 24 hours to obtain a xanthene compound in which one nitrogen atom was substituted with an aryl group.

Next, a mixture of 19.60 g (0.04 mol) of the obtained xanthene compound, 100 g of ethylene glycol, and 8.57 g (0.08 mol) of o-toluidine was heated and stirred at 120° C. for 18 hours. After completion of the reaction, the reaction solution was allowed to cool to room temperature, and the reaction solution was added dropwise to 400 g of 17.5 mass % hydrochloric acid, followed by stirring at room temperature for 1 hour. Thereafter, the precipitate was collected by filtration, washed with pure water at 80° C., and dried at 60° C. for 24 hours to obtain a xanthene compound in which two nitrogen atoms were substituted with an aryl group.

Subsequently, a mixture of 19.62 g (0.035 mol of the obtained xanthene compound, 130 g of 1-methyl-2-pyrrolidinone, 7.8 g of potassium carbonate, and 14.9 g (0.105 mol) of methyl iodide was stirred at 80° C. for 2 hours. After completion of the reaction, the reaction solution was allowed to cool to room temperature, and the reaction solution was added dropwise to 540 g of 17.5 mass % hydrochloric acid at 0 to 10° C., followed by stirring for 1 hour. Thereafter, the precipitate was collected by filtration and dried at 60° C. for 24 hours to obtain a xanthene compound (1). The obtained compound was subjected to LC-MS analysis using LC-MS2020 (manufactured by SHIMADZU CORPORATION) and confirmed to be a target compound.

LC-MS (ESI, posi): m/z 589 [M+H]⁺

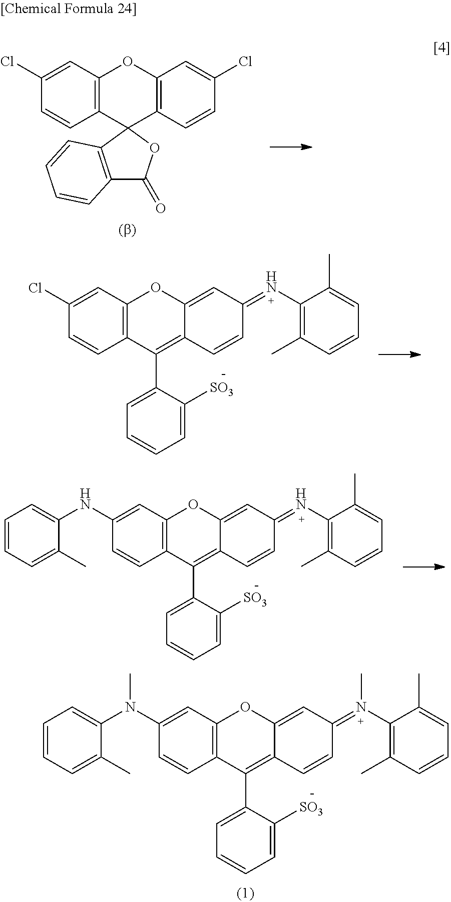

### Comparative Synthesis Example 2 Synthesis of Xanthene Compound (2)

A xanthene compound in which two nitrogen atoms were substituted with an aryl group not having an electron donating substituent was obtained in the same manner as in Synthesis Example 5 except that 20.58 g (0.15 mol) of 4-ethoxyaniline was changed to g (0.015 mol) of aniline in the following Reaction Formula [9].

Next, a xanthene compound (2) in which four nitrogen atoms were substituted with an aryl group not having an electron donating substituent was obtained in the same manner as in Synthesis Example 5 except that 22.83 g (0.04 mol) of (b-2-1) was changed to 19.30 g (0.04 mol) of the obtained xanthene compound in which two nitrogen atoms were substituted with an aryl group not having an electron donating substituent, and 19.84 g (0.08 mol) of 4-iodophenitol was changed to 16.32 g (0.08 mol) of iodobenzene.

LC-MS (ESI, posi): m/z 635 [M+H]⁺

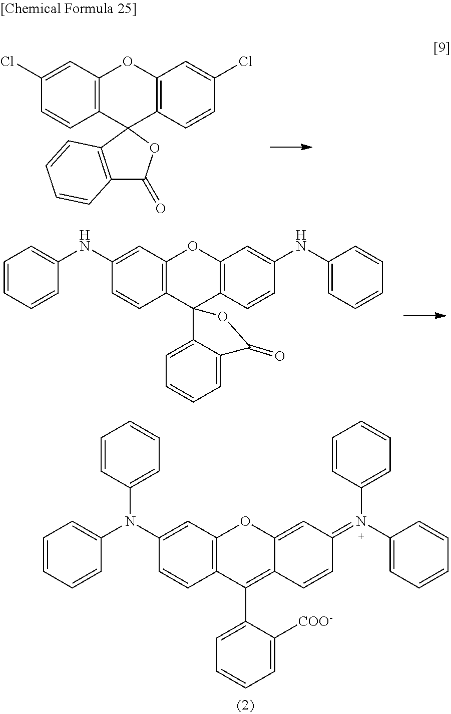

The names of the compounds used in each of Examples and Comparative Examples are shown below. The colorant (d) was synthesized using a known method, and the maximum absorption wavelength was calculated by measuring the transmission spectrum at a wavelength of 300 nm to 800 nm in the GBL solution using an ultraviolet-visible spectrophotometer MultiSpec-1500 (manufactured by SHIMADZU CORPORATION). The maximum absorption wavelength of the compound (d10-2-1) was 534 nm, and the maximum absorption wavelength of the compound (d10-2-2) was 536 nm.
e-1: 4,4′,4′-methylidyne trisphenol (thermally coloring compound)
GBL: γ-butyrolactone
EL: ethyl lactate
PGME: propylene glycol monomethyl ether

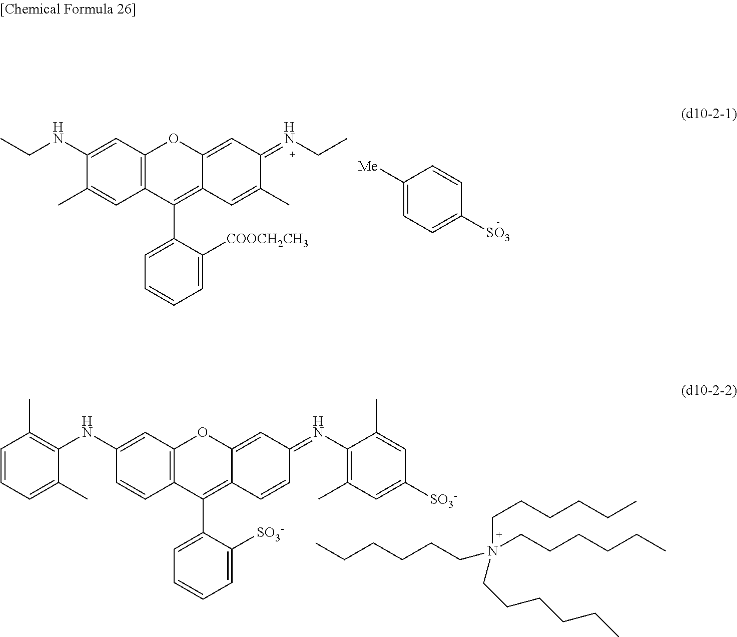

### Example 1

To 20 g of GBL, 7.0 g of the polyimide precursor (a-1) and 0.5 g of the xanthene compound (b-1) were added to obtain a varnish A1 of a resin composition containing the xanthene compound (b). To 20 g of GBL, 7.0 g of the polyimide precursor (a-1) was added to obtain a varnish B1 of a resin composition not containing the xanthene compound (b). The maximum absorption wavelength at 350 to 800 nm and the heat resistance of the dye were evaluated as described above using the obtained varnishes A1 and B1.

### Examples 2 to 11 and Comparative Examples 1 to 3

A varnish A of a resin composition containing the xanthene compound (b) and a varnish B of a resin composition not containing the xanthene compound (b) were obtained in the same manner as in Example 1 except that the alkali-soluble resin (a), the xanthene compound (b), and the solvent were changed as shown in Table 1. The maximum absorption wavelength at 350 to 800 nm and the heat resistance of the dye were evaluated as described above using the obtained varnishes A and B.

### Example 12

In 10 g of GBL, 20 g of EL, and 70 g of PGME, 10.0 g of the polyimide precursor (a-1), 2.0 g of the xanthene compound (b), 2.0 g of the photosensitive compound (c-1), 1.0 g of (d10-2-2), and 2.0 g of (e-1) were dissolved, and then the resulting solution was filtered through a 0.2 μm polytetrafluoroethylene filter to obtain a varnish AA of a positive photosensitive resin composition. The sensitivity, the OD value, and the change amount of the OD value were evaluated as described above using the obtained varnish.

### Examples 13 to 23 and Comparative Examples 3 to 5

A varnish of a positive photosensitive resin composition was obtained in the same manner as in Example 12 except that the alkali-soluble resin (a), the xanthene compound (b), the photosensitive compound (c), the coloring material (d), other additives, and the solvent were changed as shown in Table 2. The sensitivity, the OD value, and the change amount of the OD value were evaluated as described above using the obtained varnish.

### Examples 24 and 25

The frozen storage stability was evaluated as described above using the varnish of the positive photosensitive resin composition described in Table 2.

### Example 26

The xanthene compound (b′) in the cured film was analyzed by TOF-SIMS as described above using the cured film of the resin composition AE obtained in Example 16. As a result of the analysis, molecular ions of m/z 902 (⁹⁰²C₆₂H₅₂N₃O₄) were confirmed. From this result, it was confirmed that the cured film of the resin composition AE contained a cation moiety of the xanthene compound (b-5).

Tables 1 to 4 show the compositions and evaluation results in Examples and Comparative Examples.

TABLE 1

| | Varnish A | | | | | | | |
|---|---|---|---|---|---|---|---|---|
| | | | Varnish B | | Maximum absorption | | Heat resistance | |
| | | Anion moiety | Alkali- | | wavelength | | Absorbance | |
| | Xanthene | of xanthene | soluble | | Wavelength | | change rate | |
| | compound (b) | compound (b) | resin (a) | Solvent | (nm) | Determination | (%) | Determination |
| Example 1 | b-1 | — | a-1 | GBL | 583 | B | 100 | A |
| | 0.5 g | | 7.0 g | 20 g | | | | |
| Example 2 | b-2 | — | a-1 | GBL | 604 | A | 78 | B |
| | 0.5 g | | 7.0 g | 20 g | | | | |
| Example 3 | b-3 | Chlorine ion | a-1 | GBL | 622 | A | 100 | A |
| | 0.5 g | | 7.0 g | 20 g | | | | |
| Example 4 | b-4 | Chlorine ion | a-1 | GBL | 612 | A | 100 | A |
| | 0.5 g | | 7.0 g | 20 g | | | | |
| Example 5 | b-5 | p-Toluenesulfonate | a-1 | GBL | 609 | A | 100 | A |
| | 0.5 g | | 7.0 g | 20 g | | | | |
| Example 6 | b-6 | Lauryl benzenesulfonate | a-1 | GBL | 610 | A | 100 | A |
| | 0.5 g | | 7.0 g | 20 g | | | | |
| Example 7 | b-7 | Chlorine ion | a-1 | GBL | 603 | A | 88 | A |
| | 0.5 g | | 7.0 g | 20 g | | | | |
| Example 8 | b-8 | Trifluoromethanesulfonate | a-1 | GBL | 610 | A | 100 | A |
| | 0.5 g | | 7.0 g | 20 g | | | | |
| Example 9 | b-9 | Tetraphenylborate | a-1 | GBL | 610 | A | 100 | A |
| | 0.5 g | | 7.0 g | 20 g | | | | |
| Example 10 | b-10 | Bis(trifluoromethane- | a-1 | GBL | 610 | A | 100 | A |
| | 0.5 g | sulfonyl)imide ion | 7.0 g | 20 g | | | | |
| Example 11 | b-11 | Chlorine ion | a-1 | GBL | 596 | B | 100 | A |
| | 0.5 g | | 7.0 g | 20 g | | | | |
| Comparative | Xanthene | — | a-1 | GBL | 557 | C | 96 | A |
| Example 1 | compound 1 | | 7.0 g | 20 g | | | | |
| | 0.5 g | | | | | | | |
| Comparative | Basic Blue 7 | Chlorine ion | a-1 | GBL | 617 | A | 40 | C |
| Example 2 | 0.5 g | | 7.0 g | 20 g | | | | |
| Comparative | Xanthene | Chlorine ion | a-1 | GBL | 564 | C | 76 | B |
| Example 3 | compound 2 | | 7.0 g | 20 g | | | | |
| | 0.5 g | | | | | | | |

TABLE 2

| | | Alkali- | | | | | | | |
|---|---|---|---|---|---|---|---|---|---|
| | | soluble | Xanthene | Photosensitive | | | | | |
| | | resin (a) | compound | compound | Colorant | Others | | | |
| | | (Kind) | (b) (Kind) | (c) (Kind) | (d) (Kind) | (Kind) | | | |
| | Resin | (Blending | (Blending | (Blending | (Blending | (Blending | Solvent (Kind) | | |
| | composition | amount) | amount) | amount) | amount) | amount) | (Blending amount) | | |
| Example 12 | AA | a-1 | b-1 | c-1 | d10-2-2 | e-1 | GBL | EL | PGME |
| | | 10.0 g | 2.0 g | 2.0 g | 1.0 g | 2.0 g | 10 g | 20 g | 70 g |
| Example 13 | AB | a-1 | b-2 | c-1 | d10-2-2 | e-1 | GBL | EL | PGME |
| | | 10.0 g | 2.0 g | 2.0 g | 1.0 g | 2.0 g | 10 g | 20 g | 70 g |
| Example 14 | AC | a-1 | b- 3 | c-1 | d10-2-2 | e-1 | GBL | EL | PGME |
| | | 10.0 g | 2.0 g | 2.0 g | 1.0 g | 2.0 g | 10 g | 20 g | 70 g |
| Example 15 | AD | a-1 | b-4 | c-1 | d10-2-2 | e-1 | GBL | EL | PGME |
| | | 10.0 g | 2.0 g | 2.0 g | 1.0 g | 2.0 g | 10 g | 20 g | 70 g |
| Example 16 | AE | a-1 | b-5 | c-1 | d10-2-2 | e-1 | GBL | EL | PGME |
| | | 10.0 g | 2.0 g | 2.0 g | 1.0 g | 2.0 g | 10 g | 20 g | 70 g |
| Example 17 | AF | a-1 | b-6 | c-1 | d10-2-2 | e-1 | GBL | EL | PGME |
| | | 10.0 g | 2.0 g | 2.0 g | 1.0 g | 2.0 g | 10 g | 20 g | 70 g |
| Example 18 | AG | a-1 | b-7 | c-1 | d10-2-2 | e-1 | GBL | EL | PGME |
| | | 10.0 g | 2.0 g | 2.0 g | 1.0 g | 2.0 g | 10 g | 20 g | 70 g |
| Example 19 | AH | a-1 | b-5 | c-1 | d10-2-1 | e-1 | GBL | EL | PGME |
| | | 10.0 g | 2.0 g | 2.0 g | 1.0 g | 2.0 g | 10 g | 20 g | 70 g |
| Example 20 | AI | a-1 | b-8 | c-1 | d10-2-2 | e-1 | GBL | EL | PGME |
| | | 10.0 g | 2.0 g | 2.0 g | 1.0 g | 2.0 g | 10 g | 20 g | 70 g |
| Example 21 | AJ | a-1 | b-9 | c-1 | d10-2-2 | e-1 | GBL | EL | PGME |
| | | 10.0 g | 2.0 g | 2.0 g | 1.0 g | 2.0 g | 10 g | 20 g | 70 g |
| Example 22 | AK | a-1 | b-10 | c-1 | d10-2-2 | e-1 | GBL | EL | PGME |
| | | 10.0 g | 2.0 g | 2.0 g | 1.0 g | 2.0 g | 10 g | 20 g | 70 g |
| Example 23 | AK | a-1 | b-11 | c-1 | d10-2-2 | e-1 | GBL | EL | PGME |
| | | 10.0 g | 2.0 g | 2.0 g | 1.0 g | 2.0 g | 10 g | 20 g | 70 g |
| Comparative | ZA | a-1 | Xanthene compound 1 | c-1 | d10-2-2 | e-1 | GBL | EL | PGME |
| Example 3 | | 10.0 g | 2.0 g | 2.0 g | 1.0 g | 2.0 g | 10 g | 20 g | 70 g |
| Comparative | ZB | a-1 | Basic Blue 7 | c-1 | d10-2-2 | e-1 | GBL | EL | PGME |
| Example 4 | | 10.0 g | 2.0 g | 2.0 g | 1.0 g | 2.0 g | 10 g | 20 g | 70 g |
| Comparative | ZC | a-1 | Xanthene compound 2 | c-1 | d10-2-2 | e-1 | GBL | EL | PGME |
| Example 5 | | 10.0 g | 2.0 g | 2.0 g | 1.0 g | 2.0 g | 10 g | 20 g | 70 g |

TABLE 3

| | | | | | Change amount of OD value | | |
|---|---|---|---|---|---|---|---|
| | | | | | by repeated curing | | |
| | Sensitivity | | OD value per 1 μm | | | Change | |
| | (mJ/cm²) | Determination | (/μm) | Determination | (/μm) | amount | Determination |
| Example 12 | 125 | B | 0.82 | A | 0.82 | 0.00 | A |
| Example 13 | 130 | B | 0.78 | A | 0.70 | 0.08 | B |
| Example 14 | 130 | B | 0.76 | A | 0.76 | 0.00 | A |
| Example 15 | 130 | B | 0.81 | A | 0.81 | 0.00 | A |
| Example 16 | 110 | A | 0.75 | A | 0.75 | 0.00 | A |
| Example 17 | 115 | A | 0.68 | B | 0.68 | 0.00 | A |
| Example 18 | 120 | B | 0.84 | A | 0.79 | 0.05 | B |
| Example 19 | 110 | A | 0.77 | A | 0.73 | 0.04 | A |
| Example 20 | 120 | B | 0.77 | A | 0.77 | 0.00 | A |
| Example 21 | 145 | B | 0.67 | B | 0.67 | 0.00 | A |
| Example 22 | 130 | B | 0.72 | A | 0.72 | 0.00 | A |
| Example 23 | 130 | B | 0.70 | A | 0.70 | 0.00 | A |
| Comparative | 125 | B | 0.40 | C | 0.39 | 0.01 | C |
| Example 3 | | | | | | | |
| Comparative | 130 | B | 0.71 | A | 0.56 | 0.15 | C |
| Example 4 | | | | | | | |
| Comparative | 130 | B | 0.48 | C | 0.40 | 0.08 | C |
| Example 5 | | | | | | | |

TABLE 4

| | | Kind of | | |
|---|---|---|---|---|
| | Resin | organic | Frozen storage stability | |
| | composition | anion | (Number/cm²) | Determination |
| Example 24 | AE | 2 | 1.95 | B |
| Example 25 | AH | 1 | 0.04 | A |

### DESCRIPTION OF REFERENCE SIGNS

1: TFT (thin film transistor)
2: Wiring
3: TFT insulation layer
4: Planarization layer
5: ITO (transparent electrode)
6: Substrate
7: Contact hole
8: Insulation layer
11: Display device
12: Luminescent element
13: Cured object
14, 14c: Metal wiring
15: Counter substrate
16: Electrode terminal
17: Luminescent element driving substrate
18: Drive element
19: Barrier metal
20: Solder bump

1. A xanthene compound (b) represented by Formula (1): 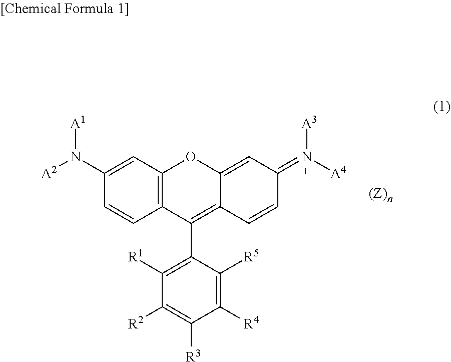
wherein A¹ to A⁴ each independently represent a hydrogen atom, an alkyl group having 1 to 10 carbon atoms, or an aryl group having 6 to 10 carbon atoms which may have an electron donating substituent, provided that, at least three of A¹ to A⁴ are the aryl groups having 6 to 10 carbon atoms which may have an electron donating substituent, and at least one of the aryl groups having 6 to 10 carbon atoms which may have an electron donating substituent has an electron donating substituent; R¹ to R⁴ each independently represent a hydrogen atom, a halogen atom, a hydroxyl group, an alkoxy group, —SO₃H, —SO₃⁻, —SO₃NR⁶R⁷, —COOH, —COO⁻, —COOR⁸, —CONR⁹R¹⁰, or a monovalent hydrocarbon group having 1 to 20 carbon atoms; R⁵ represents a hydrogen atom, —SO₃H, —SO₃⁻, —SO₃NR⁶R⁷, —COOH, —COO⁻, —COOR⁸, or —CONR⁹R¹⁰; R⁶ to R¹⁰ each independently represent a monovalent hydrocarbon group having 1 to 20 carbon atoms; and
Z represents an anionic compound, and n represents 0 or 1, provided that, the xanthene compound (b) represented by Formula (1) is charge neutral as a whole.

2. The xanthene compound (b) according to claim 1, wherein a value of a substituent constant σₚ of Hammett equation of the electron donating substituent is −0.20 or less.

3. The xanthene compound (b) according to claim 1, wherein n is 0 in the above Formula (1).

4. The xanthene compound (b) according to claim 1, wherein n is 1 and Z is an aliphatic or aromatic sulfonate ion in the above Formula (1).

5. A resin composition comprising the xanthene compound (b) according to claim 1 and an alkali-soluble resin (a).

6. The resin composition according to claim 5, further comprising a photosensitive compound (c).

7. The resin composition according to claim 6, wherein the photosensitive compound (c) contains a quinone diazide compound.

8. The resin composition according to claim 5, further comprising a colorant (d-2) having a maximum absorption wavelength in any of a range of 490 nm or more and less than 580 nm at 350 to 800 nm.

9. The resin composition according to claim 5, comprising a xanthene compound (b1) in which n is 1 and Z is an organic anion in the above Formula (1) and an ionic dye (d10) forming an ion pair of organic ions, the organic anions being one kind.

10. The resin composition according to claim 5, wherein the alkali-soluble resin (a) contains one or more selected from the group consisting of a polyimide, a polyimide precursor, a polybenzoxazole, a polybenzoxazole precursor, a polyamide-imide, a polyamide-imide precursor, and a copolymer thereof.

11. The resin composition according to claim 5, wherein a total mass of all chlorine atoms and all bromine atoms contained in the resin composition is 150 ppm or less based on a total mass of solid contents of the resin composition.

12. A cured object obtained by curing the resin composition according to claim 5.

13. A cured object comprising a xanthene compound (b′) represented by Formula (2): 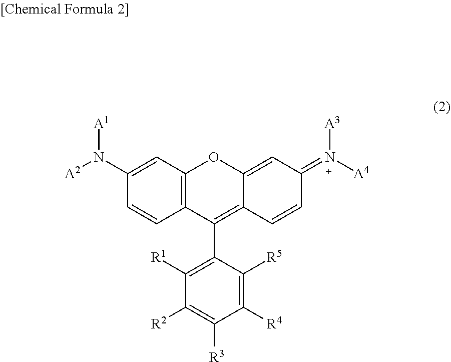
wherein A¹ to A⁴ each independently represent a hydrogen atom, an alkyl group having 1 to 10 carbon atoms, or an aryl group having 6 to 10 carbon atoms which may have an electron donating substituent, provided that, at least three of A¹ to A⁴ are the aryl groups having 6 to 10 carbon atoms which may have an electron donating substituent, and at least one of the aryl groups having 6 to 10 carbon atoms which may have an electron donating substituent has an electron donating substituent; R¹ to R⁴ each independently represent a hydrogen atom, a halogen atom, a hydroxyl group, an alkoxy group, —SO₃H, —SO₃⁻, —SO₃NR⁶R⁷, —COOH, —COO⁻, —COOR⁸, —CONR⁹R¹⁰, or a monovalent hydrocarbon group having 1 to 20 carbon atoms; R⁵ represents a hydrogen atom, —SO₃H, —SO₃⁻, —SO₃NR⁶R⁷, —COOH, —COO⁻, —COOR⁸, or —CONR⁹R¹⁰; and R⁶ to R¹⁰ each independently represent a monovalent hydrocarbon group having 1 to 20 carbon atoms, provided that, the xanthene compound (b′) represented by Formula (2) is charge neutral or cationic.

14. A method for producing a cured object, comprising the steps of:
forming a resin film formed of the resin composition according to claim 6 on a substrate;
exposing the resin film;
developing the exposed resin film; and
subjecting the developed resin film to heat treatment.

15. The method for producing a cured object according to claim 14, wherein in the step of exposing the resin film, a photomask used for exposure is a half-tone photomask having a light-transmitting portion, a light-shielding portion, and a semi-translucent portion, and a transmittance of the semi-translucent portion is 5% to 30% when a transmittance of the light-transmitting portion is taken as 100%.

16. An organic electroluminescence (EL) display device comprising:
a substrate;
a drive circuit;
a planarization layer;
a first electrode;
an insulation layer;
a light-emitting layer; and
a second electrode,
wherein the drive circuit, the planarization layer, the first electrode, the insulation layer, the light-emitting layer, and the second electrode are placed over the substrate, and the planarization layer and/or the insulation layer includes the cured object according to claim 12.

17. The organic EL display device according to claim 16, wherein the insulation layer includes the cured object, and an optical density of the insulation layer in visible light per film thickness of 1 μm is 0.5 to 1.5.

18. The organic EL display device according to claim 16, further comprising a color filter having a black matrix.

19. A display device comprising at least a metal wiring, the cured object according to claim 12, and a plurality of luminescent elements,
wherein each of the luminescent elements includes a pair of electrode terminals on either one surface, the pair of electrode terminals are connected to a plurality of the metal wirings extending in the cured object, and the plurality of the metal wirings are configured to retain electrical insulation properties by the cured object.
